(19) United States
(12) Patent Application Publication
(10) Pub. No.: US 2006/0004051 A1
(43) Pub. Date: Jan. 5, 2006

(54) Compounds

(76) Inventors: Nerina Dodic, Les Ulis (FR); Francoise Jeanne Gellibert, Les Ulis (FR)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/522,968

(22) PCT Filed: Jul. 29, 2003
(86) PCT No.: PCT/EP03/08385

§ 371 Date: Jan. 31, 2005

(30) Foreign Application Priority Data
Jul. 31, 2002 (GB) 0217787.1

(51) Int. Cl. C07D 417/14; A61K 31/5377; A61K 31/496; A61K 31/444

(52) U.S. Cl. 514/322; 514/235.5; 544/124; 546/256; 514/253.09; 544/360

(57) ABSTRACT

The invention relates to novel aminothiazole derivatives which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

This invention relates to novel aminothiazole derivatives which are inhibitors of the transforming growth factor, (“TGF”)-β signalling pathway, in particular, the phosphorylation of smad2 or smad3 by the TGF-β type I or activin-like kinase (“ALK”)-5 receptor, methods for their preparation and their use in medicine, specifically in the treatment and prevention of a disease state mediated by this pathway.

TGF-β1 is the prototypic member of a family of cytokines including the TGF-βs, activins, inhibins, bone morphogenetic proteins and Müllerian-inhibiting substance, that signal through a family of single transmembrane serine/threonine kinase receptors. These receptors can be divided into two classes, the type I or activin like kinase (ALK) receptors and type II receptors. The ALK receptors are distinguished from the type II receptors in that the ALK receptors (a) lack the serine/threonine rich intracellular tail, (b) possess serine/threonine kinase domains that are very homologous between type I receptors, and (c) share a common sequence motif called the GS domain, consisting of a region rich in glycine and serine residues. The GS domain is at the amino terminal end of the intracellular kinase domain and is critical for activation by the type II receptor. Several studies have shown that TGF-β signalling requires both the ALK and type II receptors. Specifically, the type II receptor phosphorylates the GS domain of the type I receptor for TGF-β, ALK5, in the presence of TGF-β. The ALK5, in turn, phosphorylates the cytoplasmic proteins smad2 and smad3 at two carboxy terminal serines. The phosphorylated smad proteins translocate into the nucleus and activate genes that contribute to the production of extracellular matrix. Therefore, preferred compounds of this invention are selective in that they inhibit the type I receptor and thus matrix production.

Surprisingly, it has now been discovered that a class of aminothiazole derivatives function as potent and selective non-peptide inhibitors of ALK5 kinase.

According to a first aspect, the invention provides a compound of formula (I), a pharmaceutically acceptable salt, solvate or derivative thereof: 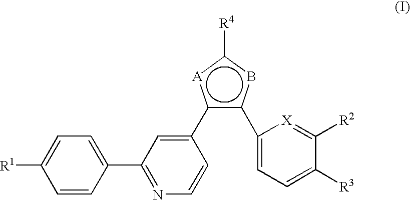
wherein
either A is S and B is N, or A is N and B is S;
X is N or CH;
R¹ is selected from hydrogen, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁸, —(CH₂)ₙNR⁵R⁶, —O(CH₂)ₙOR⁷, —O(CH₂)ₙ-Het, —O(CH₂)ₙNR⁵R⁶, —CONR⁵R⁶, —CO(CH₂)ₙNR⁵R⁶, —SO₂R⁷, —SO₂NR⁵R⁶, —NR⁵SO₂R⁷, —NR⁵COR⁷ and —O(CH₂)ₙCONR⁵R⁶;
R² is hydrogen, C₁₋₆alkyl, halo, cyano or perfluoroC₁₋₆alkyl;
R³ is hydrogen or halo;
R⁴ is —NH₂;
   where
R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4,5 or 6-membered saturated ring); or R⁵ and R⁶ together with the nitrogen atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), cyano, —CF₃, hydroxy, —OCF₃, C₁₋₆alkyl and C₁₋₆alkoxy;
R⁷ is hydrogen or C₁₋₆alkyl;
Het is a 5 or 6-membered C-linked heterocyclyl group which may be saturated, unsaturated or aromatic which may contain one or more heteroatoms selected from N, S or O and which may be substituted by C₁₋₆alkyl; and
n is 1-4;
with the proviso that the compound of formula (I) is not:
5-[2-(4-chlorophenyl)pyridin-4-yl]-4-pyridin-2-yl-1,3-thiazol-2-amine;
5-[2-(4-methoxyphenyl)pyridin-4-yl]-4-pyridin-2-yl-1,3-thiazol-2-amine;
5-[2-(4-fluorophenyl)pyridin-4-yl]-4-pyridin-2-yl-1,3-thiazol-2-amine;
5-[2-(4-ethylphenyl)pyridin-4-yl]-4-pyridin-2-yl-1,3thiazol-2-amine; or
5-[2-(4-ethoxyphenyl)pyridin-4-yl]-4-pyridin-2-yl-1,3-thiazol-2-amine.

The term “C₁₋₆alkyl” as used herein, whether on its own or as part of a group, refers to a straight or branched chain saturated aliphatic hydrocarbon radical of 1 to 6 carbon atoms, unless the chain length is limited thereto, including, but not limited to methyl, ethyl, n-propyl, isopropyl, n-butyl, sec-butyl, isobutyl, tert-butyl, pentyl and hexyl.

The term “alkenyl” as a group or part of a group refers to a straight or branched chain mono- or poly-unsaturated aliphatic hydrocarbon radical containing the specified number(s) of carbon atoms. References to “alkenyl” groups include groups which may be in the E- or Z-form or mixtures thereof.

The term “alkoxy” as a group or part of a group refers to an alkyl ether radical, wherein the term “alkyl” is defined above. Such alkoxy groups in particular include methoxy, ethoxy, n-propoxy, isopropoxy, n-butoxy, isobutoxy, sec-butoxy and tert-butoxy.

The term “perfluoroalkyl” as used herein includes compounds such as trifluoromethyl.

The term “perfluoroalkoxy” as used herein includes compounds such as trifluoromethoxy.

The terms “halo” or “halogen” are used interchangeably herein to mean radicals derived from the elements chlorine, fluorine, iodine and bromine.

The term “heterocyclyl” as used herein includes cyclic groups containing 5 to 7 ring-atoms up to 4 of which may be hetero-atoms such as nitrogen, oxygen and sulfur, and may be saturated, unsaturated or aromatic. Examples of heterocyclyl groups are furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl,thiazepinyl, diazepinyl and thiazolinyl. In addition, the term heterocyclyl includes fused heterocyclyl groups, for example benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl and isoindolyl.

Preferably X is N.

Preferably R¹ is —NR⁵R⁶, —(CH₂)ₙNR⁵R⁶, —O(CH₂)ₙ-Het (wherein Het is preferably imidazolyl or oxazolyl), —O(CH₂)ₙNR⁵R⁶, —CONR⁵R⁶, —SO₂R⁷ or —O(CH₂)ₙCONR⁵R⁶.

Preferably R⁵ and R⁶ are independently selected from hydrogen; Het (preferably tetrahydropyranyl); C₃₋₆cycloalkyl optionally substituted by C₁₋₆alkyl; or by C₁₋₆alkyl optionally substituted by Het (preferably furyl), alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy.

More preferably R¹ is morpholinyl, methanesulfonyl, 4-ethylpiperazin-1-yl, (morpholin-4-yl)carbonyl, (tetrahydropyran-4-yl)-aminocarbonyl, (morpholin-4-yl)methyl, aminocarbonylmethyloxy, 2-(pyrrolidin-1-yl)-ethoxy, (1-methyl-imidazol-4-yl)methyloxy, ethanesulfonyl, 4-(1-ethyl-piperazin-4-yl)carbonyl, (morpholin-4-yl)carbonylmethyloxy, (pyrolidin-1-yl)methyl, (dimethylamino)methyl, isopropylaminomethyl, cyclobutylaminomethyl, (5-methyl-isoxazol-3-yl)methoxy, (3,5-dimethylisoxazol-4-yl)methoxy, N-methyl-N-(3-dimethylaminopropyl)aminocarbonyl, 4-(1-isopropyl-piperazin-4-yl)carbonyl, 2-(pyrrolidin-1-yl)ethylaminocarbonyl, 3-methoxypropylaminocarbonyl, 2-(diethylamino)ethylaminocarbonyl, (2-methoxy-1-methyl)ethylaminocarbonyl, (tetrahydrofuran-2-yl)methylaminocarbonyl, 2-methoxyethylaminocarbonyl, 2-cyanoethylaminocarbonyl, (N-methyl-N-cyclohexyl)aminocarbonyl or 4-methyl-piperidin-1-ylcarbonyl.

Still more preferably R¹ is (tetrahydropyran-4-yl)-aminocarbonyl, (pyrolidin-1-yl)methyl, (dimethylamino)methyl, (morpholin-4-yl)methyl, morpholin-4-yl, 4-ethylpiperazin-1-yl or aminocarbonylmethyloxy.

Preferably R² is hydrogen, C₁₋₆alkyl, chloro or fluoro. More preferably R² is hydrogen, methyl, chloro or fluoro. More preferably still, R² is methyl.

Preferably R³ is hydrogen or fluoro.

Preferably, when X is N, R² is methyl. More preferably when X is N and R² is methyl, R³ is H.

It will be appreciated that the present invention is intended to include compounds having any combination of the preferred groups listed hereinbefore.

Preferably
either A is S and B is N, or A is N and B is S;
X is N;
R¹ is —NR⁵R⁶, —(CH₂)ₙNR⁵R⁶, —O(CH₂)ₙ-Het (wherein Het is preferably imidazolyl or oxazolyl), —O(CH₂)ₙNR⁵R⁶, —CONR⁵R⁶, —SO₂R⁷ or —O(CH₂)ₙCONR⁵R⁶;
R² is hydrogen, methyl, chloro or fluoro;
R³ is hydrogen or halo;
R⁴ is —NH₂;
   where
R⁵ and R⁶ are independently selected from hydrogen; Het (preferably tetrahydropyranyl); C₃₋₆cycloalkyl optionally substituted by C₁₋₆alkyl; or by C₁₋₆alkyl optionally substituted by Het (preferably furyl), alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy;
R⁷ is hydrogen or C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4.

According to a second aspect, the invention provides a compound as defined in the first aspect with the proviso that when A is S; B is N; X is N; R¹ is hydrogen, C₁₋₆alkyl, C₁₋₆alkoxy, halo, cyano, perfluoroC₁₋₆alkyl or perfluoroC₁₋₆alkoxy; R² is hydrogen, C₁₋₆alkyl, halo, cyano or perfluoroC₁₋₆alkyl; and R³ is hydrogen or halo; then R⁴ is not NH₂.
Compounds of formula (I) which are of special interest as agents useful in the treatment or prophylaxis of disorders characterised by the overexpression of TGF-β are selected from the list:
5-{2-[4-(4-ethylpiperazin-1-yl)phenyl]pyridin-4-yl}-4-(6-methylpyridin-2-yl)-1,3-thiazol-2-amine (Example 3);
5-{2-[4-(morpholin-4-yl)phenyl]pyridin-4-yl}-4-(6-methylpyridin-2-yl)-1,3-thiazol-2-amine (Example 4);
5-{2-[4-(aminocarbonylmethyloxy)-phenyl]pyrdin-4-yl)}-4-(6-methylpyridin-2-yl)-1,3-thiazol-2-amine (Example 10);
5-{2-[4-(2-(pyrrolidin-1-yl)-ethoxy)-phenyl]pyridin-4-yl}-4-(6-methylpyridin-2-yl)-1,3-thiazol-2-amine (Example 11);
4-[2-(4-((tetrahydropyran-4-yl)-aminocarbonyl)phenyl)pyridin-4-yl]-5-[6-methylpyridin-2-yl]-1,3-thiazol-2-amine (Example 16);
4-[2-(4-(morpholin-4-yl)phenyl)pyridin-4-yl]-5-[6-methylpyridin-2-yl]-1,3-thiazol-2-amine (Example 20);
4-[2-(4-(aminocarbonylmethyloxy)phenyl)pyridin-4-yl]-5-[6-methylpyridin-2-yl]-1,3-thiazol-2-amine (Example 22);
4-[2-(4-((pyrrolidin-1-yl)methyl)phenyl)pyridin-4-yl]-5-[6-methylpyridin-2-yl]-1,3-thiazol-2-amine (Example 24);
4-[2-(4((dimethylamino)methyl)phenyl)pyridin-4-yl]-5-[6-methylpyridin-2-yl]-1,3-thiazol-2-amine (Example 25); and
4-[2-(4-((tetrahydropyran-4-yl)aminocarbonyl)phenyl)pyridin-4-yl]-5-[pyridin-2-yl]-1,3-thiazol-2-amine (Example 26);
and pharmaceutically acceptable salts, solvates and derivatives thereof.

For the avoidance of doubt, unless otherwise indicated, the term substituted means substituted by one or more defined groups. In the case where groups may be selected from a number of alternative groups, the selected groups may be the same or different.

For the avoidance of doubt, the term independently means that where more than one substituent is selected from a number of possible substituents, those substituents may be the same or different.

As used herein the term “pharmaceutically acceptable derivative” means any pharmaceutically acceptable salt, solvate, ester or amide, or salt or solvate of such ester or amide, of the compound of formula (I), or any other compound which upon administration to the recipient is capable of providing (directly or indirectly) the a compound of formula (I) or an active metabolite or residue thereof, e.g., a prodrug. Preferred pharmaceutically acceptable derivatives according to the invention are any pharmaceutically acceptable salts, solvates or prodrugs.

Suitable pharmaceutically acceptable salts of the compounds of formula (I) include acid salts, for example sodium, potassium, calcium, magnesium and tetraalkylammonium and the like, or mono- or di-basic salts with the appropriate acid for example organic carboxylic acids such as acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solverits such as aqueous and organic solvents. In such cases solvates may be formed. This invention includes within its scope stoichiiometric solvates including hydrates as well as compounds containing variable amounts of water that may be produced by processes such as lyophilisation.

Hereinafter, compounds, their pharmaceutically acceptable salts, their solvates and polymorphs, defined in any aspect of the invention (except intermediate compounds in chemical processes) are referred to as “compounds of the invention”.

The compounds of the invention may exist in one or more tautomeric forms. All tautomers and mixtures thereof are included in the scope of the present invention.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Compounds of the invention may be prepared, in known manner in a variety of ways. In the following reaction schemes and hereafter, unless otherwise stated R¹ to R⁷, X and n are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) eta . . . (IVa), (IVb)., (IVc) etc.

Compounds of formula (la), i.e. compounds of general formula (I) where A is S, B is N and R⁴ is NH₂, may be prepared by reacting compounds of formula (II) with a suitable polymer-supported bromine reagent, such as polymer-supported pyridinium perbromide, followed by treatment with thiourea in a suitable solvent such as ethanol, preferably at elevated temperatures (see reaction scheme 1).

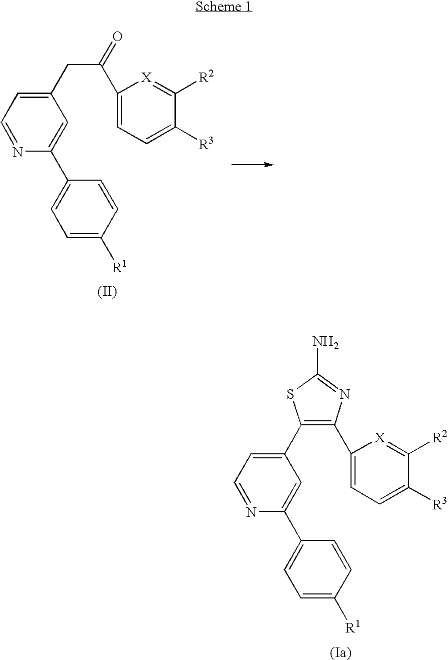

Compounds of formula (Ib), i.e. compounds of general formula (I) where A is N, B is S and R⁴ is NH₂, may be prepared by reacting compounds of formula (III) under analogous conditions to reaction scheme 1 (see reaction scheme 2).

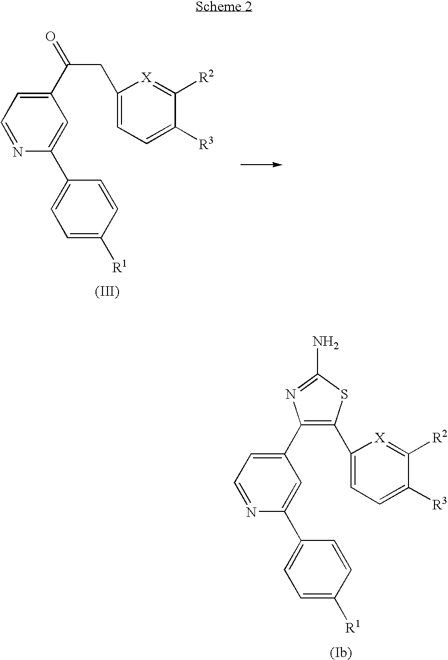

Compounds of formula (II) may be prepared by reacting compounds of formula (IV) with compounds of formula (V) where Y is a boron containing moiety such as —B(OH)₂ or 4,4,5,5-tetramethyl-1,3,2-dioxaborolan-2-yl according to reaction scheme 3. Preferred conditions comprise reaction with a suitable catalyst such as tetrakis(triphenylphosphine) palladium (O), in the presence of a suitable base such as sodium carbonate in a suitable solvent such as DME at elevated temperature.

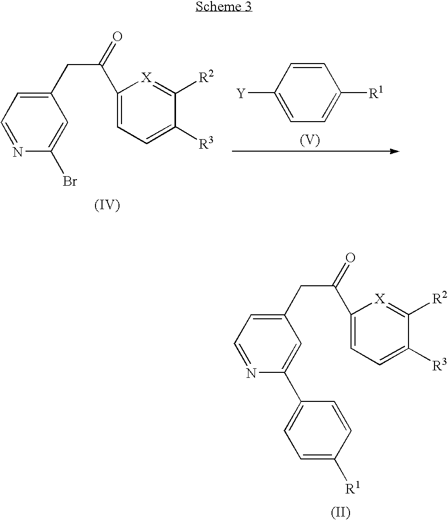

Alternatively compounds of formula (II) may be prepared by reacting compounds of formula (VI) with compounds of formula (VII) according to reaction scheme 4. Preferred reaction conditions comprise reacting (VI) with sodium bis-(trimethylsilyl)amide in a suitable solvent such as tetrahydrofuran at low temperature, preferably −78° C.

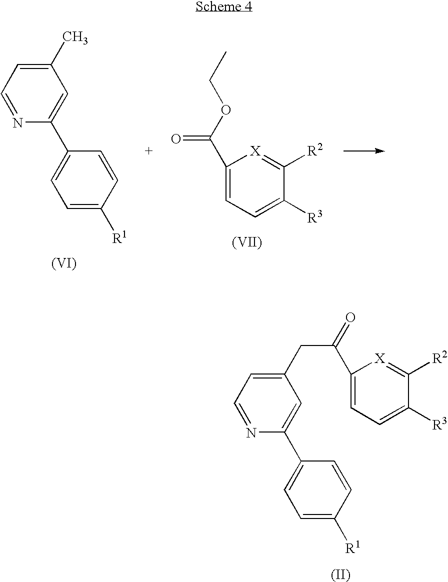

Compounds of formula (III) may be prepared according to reaction scheme 5 by reacting compounds of formula (VIII with compounds of formula (V) (where Y is as defined for reaction scheme 3) using analogous reaction conditions to those of reaction scheme 3.

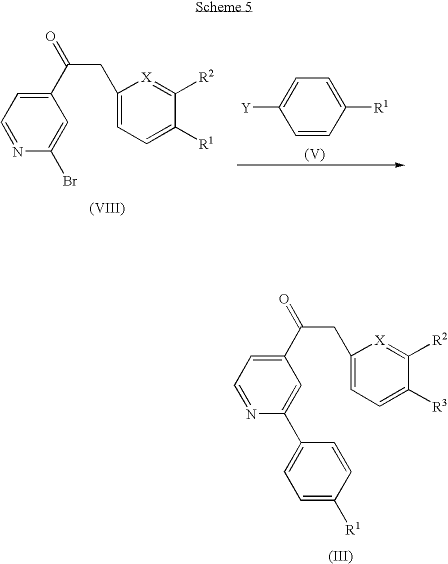

Alternatively compounds of formula (III) may be prepared according to reaction scheme 6 by reacting compounds of formula (IX) with compounds of formula (X) in the presence of a suitable base such as cesium carbonate in a suitable solvent such as tetrahydrofuran and isopropanol at room temperature.

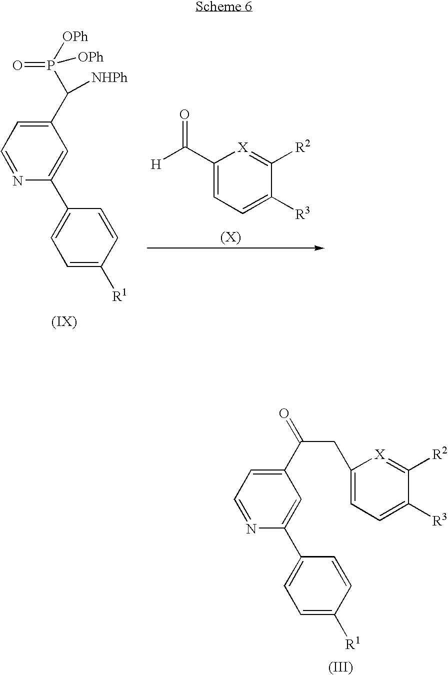

Compounds of formula (VIIIa), i.e. compounds of general formula (VIII) (see scheme 5) where X is N, R² is methyl and R³ is hydrogen, may be prepared according to reaction scheme 7. Preferred conditions comprise reacting 2,6-lutidine with a strong base such as n-butyllithium or sodium bis-(trimethylsilyl) amide at low temperature, followed by addition of 2-bromo-N-methoxy-N-methyl-4-pyridinecarboxamide (see preparation of Intermediate 36 hereinafter)

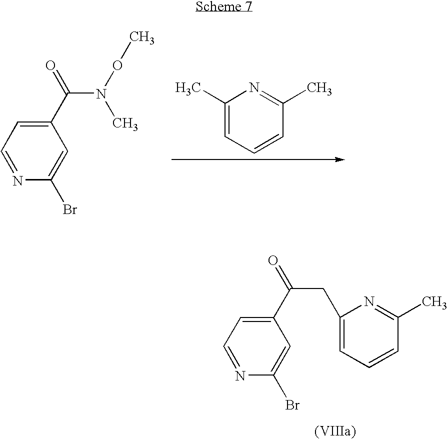

Compounds of formula (IX) may be prepared in two steps according to reaction scheme 8. Preferred reaction conditions for the first step are analogous to those described for reaction scheme 3. Preferred reaction conditions for the second step comprise reacting compounds of formula (XI) with aniline and diphenylphosphite in a suitable solvent such as isopropanol at room temperature.

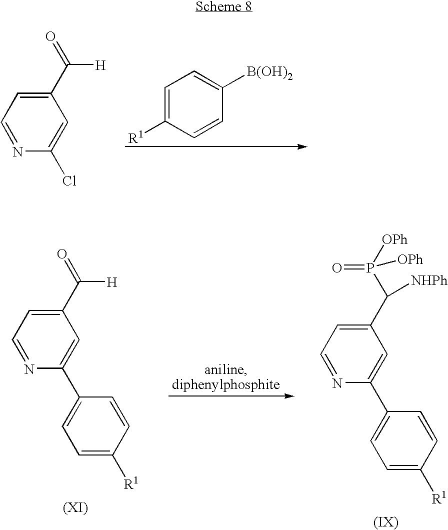

Compounds of general formula (I) may also be prepared using solid supported chemistry.

Compounds of formula (Ic), i.e. compounds of general formula (I) where A is S, B is N, R¹ is —OR (where R is for example —(CH₂)ₙ-Het or —CH₂CONR⁵R⁶) and R⁴ is NH₂, may be prepared from solid supported compounds of formula (XII) by reaction with RX (where X is a suitable leaving group such as chlorine) followed by cleavage under acidic conditions from the solid support, according to reaction scheme 9. Preferred conditions comprise treating (XII) with RX under basic conditions such as potassium carbonate in a suitable solvent such as DMSO at elevated temperature. Preferred cleavage conditions are trifluoroacetic acid in a suitable solvent such as dichloromethane at room temperature.

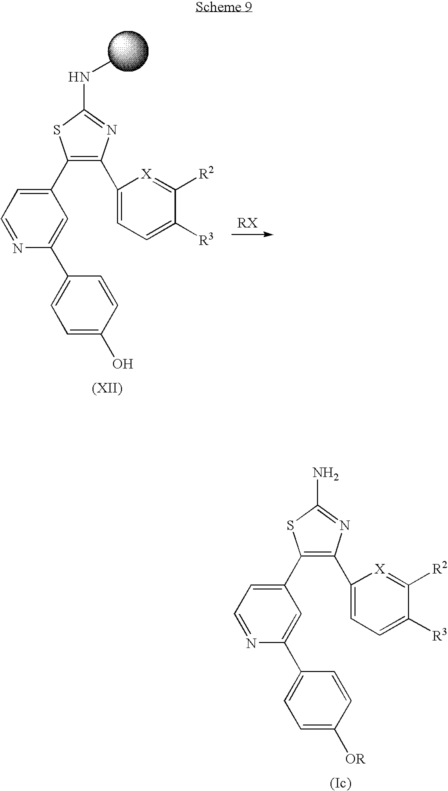

Compounds of formula (Id), i.e. compounds of general formula (I) where A is S, B is N, R¹ is —CH₂NR⁵R⁶ and R⁴ is NH₂, may :be prepared from solid supported compounds of formula (XIII) according to reaction scheme 10. Preferred reaction conditions comprise treating (XIII) with HNR⁵R⁶ in trimethylorthoformate and addition of a reducing agent, such as sodium cyanoborohydride in acetic acid at elevated temperature. Cleavage from the solid support using trifluoroacetic acid in dichloromethane gives compounds of formula (Id).

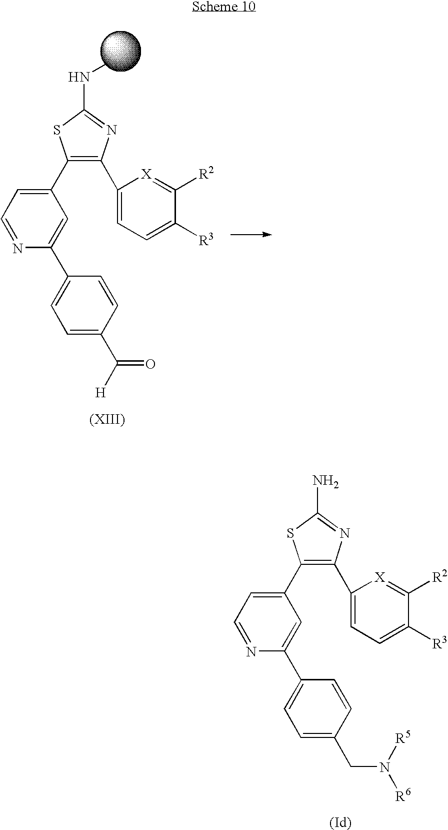

Compounds of formula (Ie), i.e. compounds of general formula (I) where A is S, B is N, R¹ is —C(O)NR⁵R⁶ and R⁴ is NH₂, may be prepared from solid supported compounds of formula (XIV) according to reaction scheme 11. Preferred reaction conditions comprise treating (XIV) with HNR⁵R⁶, hydroxybenzotriazole and diisopropylcarbodiimide. Cleavage from the solid support using trifluoroacetic acid in dichloromethane gives compounds of formula (Ie).

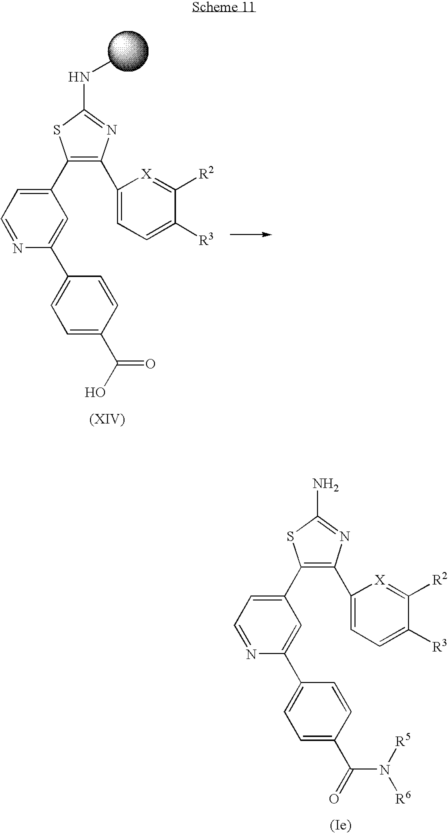

Compounds of formula (XII), (XIII) and (XIV) may be prepared according to reaction scheme 12 from compounds of formula (XV) and the appropriate arylboronic acid (XVI), where Z is —OH, —CHO or —CO₂H respectively.

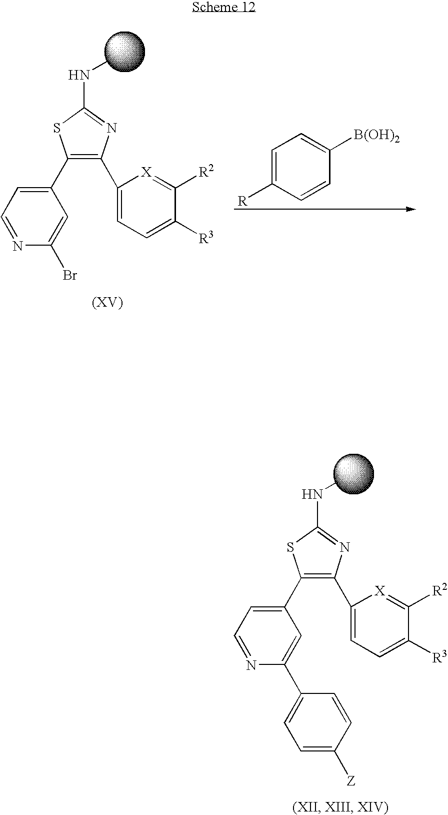

Compounds of formula (XV) may be prepared by solid phase synthesis according to reaction scheme 13. Compounds of formula (XVI) may be prepared by treating compounds of formula (IV) (see scheme 3) with a suitable polymer-supported bromine reagent, such as polymer-supported pyridinium perbromide. Treatment of a resin-bound thiourea with a dioxane solution of compounds of formula (XVI) gives compounds of formula (XV) using general conditions described in the literature (see Kearney P. C., J. Org. Chem., (1998), 63, 196).

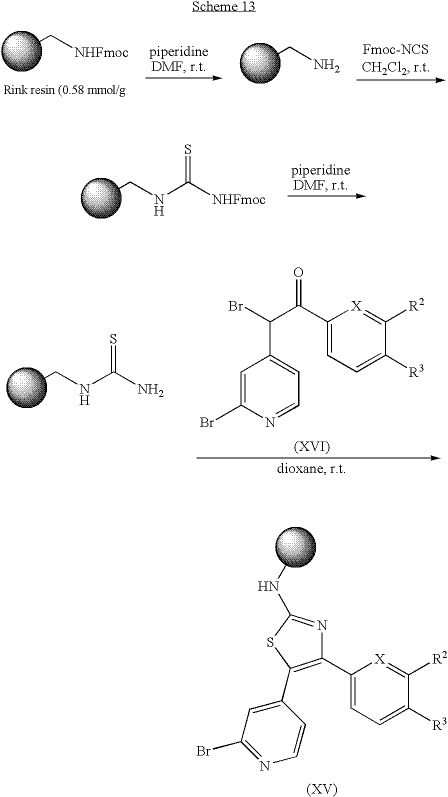

Further details for the preparation of compounds of formula (I) are found in the examples.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Activation of the TGF-β1 axis and expansion of extracellular matrix are early and persistent contributors to the development and progression of chronic renal disease and vascular disease. Border W. A, et al, N. Engl. J. Med., 1994; 331(19),1286-92. Further, TGF-β1 plays a role in the formation of fibronectin and plasminogen activator inhibitor-1, components of sclerotic deposits, through the action of smad3 phosphorylation by the TGF-β1 receptor ALK5. Zhang Y., et al, Nature, 1998; 394(6696), 909-13; Usui T., et al, Invest. Ophthalmol. Vis. Sci., 1998; 39(11), 1 981-9.

Progressive fibrosis in the kidney and cardiovascular system is a major cause of suffering and death and an important contributor to the cost of health care. TGF-β1 has been implicated in many renal fibrotic disorders. Border W. A., et al, N. Engl. J. Med., 1994; 331(19),1286-92. TGF-β1 is elevated in acute and chronic glomerulonephritis Yoshioka K., et al, Lab. Invest., 1993; 68(2), 154-63, diabetic nephropathy Yamamoto, T., et al, 1993, PNAS 90, 1814-1818, allograft rejection, HIV nephropathy and angiotensin-induced nephropathy Border W. A., et al, N. Engl. J. Med., 1994; 331(19),1286-92. In these diseases the levels of TGF-β1 expression coincide with the production of extracellular matrix. Three lines of evidence suggest a causal relationship between TGF-β1 and the production of matrix. First, normal glomeruli, mesangial cells and non-renal cells can be induced to produce extracellular-matrix protein and inhibit protease activity by exogenous TGF-β1 in vitro. Second, neutralizing anti-bodies against TGF-β1 can prevent the accumulation of extracellular matrix in nephritic rats Third, TGF-β1 transgenic mice or in vivo transfection of the TGF-β1 gene into normal rat kidneys resulted in the rapid development of glomerulosclerosis. Kopp J. B., et al, Lab. Invest, 1996; 74(6), 991-1003. Thus, inhibition of TGF-β1 activity is indicated as a therapeutic intervention in chronic renal disease.

TGF-β1 and its receptors are increased in injured blood vessels and are indicated in neointima formation following balloon angioplasty Saltis J., et al, Clin. Exp. Pharmacol. Physiol., 1996; 23(3),193-200. In addition TGF-β1 is a potent stimulator of smooth muscle cell (“SMC”) migration in vitro and migration of SMC in the arterial wall is a contributing factor in the pathogenesis of atherosclerosis and restenosis. Moreover, in multivariate analysis of the endothelial cell products against total cholesterol, TGF-β receptor ALK5 correlated with total cholesterol (P<0.001) Blann A. D., et al, Atherosclerosis, 1996; 120(1-2), 221-6. Furthermore, SMC derived from human atherosclerotic lesions have an increased ALK5/TGF-β type II receptor ratio. Because TGF-β1 is over-expressed in fibroproliferative vascular lesions, receptor-variant cells would be allowed to grow in a slow, but uncontrolled fashion, while overproducing extracellular matrix components McCaffrey T. A., et al, Jr., J. Clin. Invest., 1995; 96(6), 2667-75. TGF-β1 was immunolocalized to non-foamy macrophages in atherosclerotic lesions where active matrix synthesis occurs, suggesting that non-foamy macrophages may participate in modulating matrix gene expression in atherosclerotic remodelling via a TGF-β-dependent mechanism. Therefore, inhibiting the action of TGF-β1 on ALK5 is also indicated in atherosclerosis and restenosis.

TGF-β is also indicated in wound repair. Neutralizing antibodies to TGF-β1 have been used in a number of models to illustrate that inhibition of TGF-β1 signalling is beneficial in restoring function after injury by limiting excessive scar formation during the healing process. For example, neutralizing antibodies to TGF-β1 and TGF-β2 reduced scar formation and improved the cytoarchitecture of the neodermis by reducing the number of monocytes and macrophages as well as decreasing dermal fibronectin and collagen deposition in rats Shah M., J. Cell. Sci., 1995, 108, 985-1002. Moreover, TGF-β antibodies also improve healing of corneal wounds in rabbits Moller-Pedersen T., Curr. Eye Res., 1998, 17, 736-747, and accelerate wound healing of gastric ulcers in the rat, Ernst H., Gut, 1996, 39, 172-175. These data strongly suggest that limiting the activity of TGF-β would be beneficial in many tissues and suggest that any disease with chronic elevation of TGF-β would benefit by inhibiting smad2 and smad3 signalling pathways.

TGF-β is also implicated in peritoneal adhesions Saed G. M., et al, Wound Repair Regeneration, 1999 November-December, 7(6), 504-510. Therefore, inhibitors of ALK5 would be beneficial in preventing peritoneal and sub-dermal fibrotic adhesions following surgical procedures.

TGF-β is also implicated in photoaging of the skin (see Fisher G J. Kang S W. Varani J. Bata-Csorgo Z Wan Y S. Data S. Voorhees J J. , Mechanisms of photoaging and chronological skin ageing, Archives of Dermatology, 138(11):1462-1470, 2002 November and Schwartz E. Sapadin A N. Kligman L H. “Ultraviolet B radiation increases steady state mRNA levels for cytokines and integrins in hairless mouse skin-modulation by topical tretinoin”, Archives if Dermatological Research, 290(3):137-144, 1998 March)

Therefore according to a further aspect, the invention provides the use of a compound defined in the first aspect in the preparation of a medicament for treating or preventing a disease or condition mediated by ALK-5 inhibition.

Preferably the disease or condition mediated by ALK-5 inhibition is selected from the list: chronic renal disease, acute renal disease, wound healing, arthritis, osteoporosis, kidney disease, congestive heart failure, ulcers (including diabetic ulcers, chronic ulcers, gastric ulcers, and duodenal ulcers), ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to kidney fibrosis, lung fibrosis and liver fibrosis, for example, hepatitis B virus (HBV), hepatitis C virus (HCV), alcohol-induced hepatitis, haemochromatosis, primary biliary cirrhosis, restenosis, retroperitoneal fibrosis, mesenteric fibrosis, endometriosis, keloids, cancer, abnormal bone function, inflammatory disorders, scarring and photaging of the skin.

More preferably the disease or condition mediated by ALK-5 inhibition is fibrosis. Preferably kidney fibrosis.

It will be appreciated that references herein to treatment extend to prophylaxis as well as the treatment of established conditions.

Compounds of the invention may be administered in combination with other therapeutic agents, for example antiviral agents for liver diseases, or in combination with ACE inhibitors or angiotensin II receptor antagonists for kidney diseases.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and steirilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

No toxicological effects are indicated when a compound of the invention is administered in the above-mentioned dosage range.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The preferred embodiments described for the first aspect extend these further aspects:

i) a pharmaceutical composition comprising a compound of the invention and a pharmaceutically acceptable carrier or diluent;

ii) a compound of the invention for use as a medicament;

iii) a method of treatment or prophylaxis of a disorder selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis [for example, hepatitis B virus (HBV), hepatitis C virus (HCV)], alcohol induced hepatitis, retroperitoneal fibrosis, mesenteric fibrosis, haemochromatosis and primary biliary cirrhosis, endometriosis, keloids, restenosis and photoaging of the skin, in mammals, which comprises administration to the mammal in need of such treatment, an effective amount of a compound of the invention; and

iv) a combination of a compound of the invention with an ACE inhibitor or an angiotensin II receptor antagonist.

According to a further aspect, the invention provides a compound of formula (I), a pharmaceutically acceptable salt, solvate or derivative thereof; 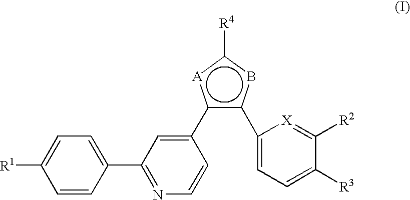
wherein
A is S and B is N;
X is N or CH;
R¹ is selected from H, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁶, —(CH₂)ₙNR⁵R⁶, —O(CH₂)ₙOR⁷, —O(CH₂)ₙNR⁵R⁶, —CONR⁵R⁶, —CO(CH₂)ₙNR⁵R⁶, —SO₂R⁷, —SO₂NR⁵R⁶, —NR⁵SO₂R⁷ and —NR⁵COR⁷;
R² is selected from H, C₁₋₆alkyl, halo, CN or perfluoroC₁₋₆alkyl;
R³ is selected from H or halo;
R⁴ is selected from —NR⁵R⁶;
R⁵, R⁶ and R⁷ are independently selected from H or C₁₋₆alkyl; or R⁵ and R⁶ together with the atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, —OCF₃, C₁₋₆ alkyl and C₁₋₆ alkoxy; and
n is 1-4;
with the proviso that the compound of formula (I) is not:
5-[2-(4-chlorophenyl)pyridin-4-yl]-4-pyridin-2-yl-1,3-thiazol-2-amine;
5-[2-(4-methoxyphenyl)pyridin-4-yl]-4-pyridin-2-yl-1,3-thiazol-2-amine;
5-[2-(4-fluorophenyl)pyridin-4-yl]-4-pyridin-2-yl-1,3-thiazol-2-amine;
5-[2-(4-ethylphenyl)pyridin-4-yl]-4-pyridin-2-yl-1,3-thiazol-2-amine; or
5-[2-(4-ethoxyphenyl)pyridin-4-yl]-4-pyridin-2-yl-1,3-thiazol-2-amine.

According to a still further aspect, the invention provides a compound of formula (I), a pharmaceutically acceptable salt, solvate or derivative thereof;

wherein

A is S and B is N;
X is N or CH;
R¹ is selected from H, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁶, —(CH₂)ₙNR⁵R⁶, —O(CH₂)ₙOR⁷, —O(CH₂)ₙNR⁵R⁶, —CONR⁵R⁶, —CO(CH₂)ₙNR⁵R⁶, —SO₂R⁷, —SO₂NR⁵R⁶, —NR⁵SO₂R⁷ and —NR⁵COR⁷;
R² is selected from H, C₁₋₆alkyl, halo, CN or perfluoroC₁₋₆alkyl;
R³ is selected from H or halo;
R⁴ is selected from —NR⁵R⁶;
R⁵, R⁶ and R⁷ are independently selected from H or C₁₋₆alkyl; or R⁵ and R⁶ together with the atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), —CN, —CF₃, —OH, OCF₃, C₁₋₆ alkyl and C₁₋₆ alkoxy; and
n is 1-4;
with the proviso that when A is S; B is N; X is N; R¹ is hydrogen, C₁₋₆alkyl, C₁₋₆alkoxy, halo, cyano, perfluoroC₁₋₆alkyl or perfluoroC₁₋₆alkoxy; R² is hydrogen, C₁₋₆alkyl, halo, cyano or perfluoroC₁₋₆alkyl; and R³ is hydrogen or halo; then R⁴ is not NH₂.

The following non-limiting examples illustrate the present invention.

Abbreviations

Binap 2,2′-Bis(diphenylphosphino)-1,1′-binaphthyl
CH₂Cl₂ dichloromethane
DMF dimethylformamide
DME 1,2-Dimethoxyethane
DMSO dimethylsulfoxide
EDCl 1-[3-(Dimethylamino)propyl]-3-ethylcarbodiimide hydrochloride
EtOAc ethyl acetate
Et₂O diethyl oxide
EtOH ethanol
Et₃N triethylamine
Fmoc-NCS fluoromethylcarbonyl isothiocyanate
HOBT hydroxybenzotriazole
MeOH methanol
NaH sodium hydride
NaHCO₃ sodium hydrogen carbonate
NaHMDS sodium bis(trimethylsilyl)amide
NH₄Cl ammonium chloride
Na₂SO₄ sodium sulfate
Pd₂(dba)₃ tris(dibenzylideneacetone)dipalladium(0)
Pd(PPh₃)₄ tetrakis(triphenylphosphine)palladium (0)
THF tetrahydrofuran

### Intermediate 1: 1-methyl-4-hydroxymethyl-imidazole

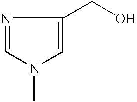

To a suspension of 1-methyl-imidazole-4-carboxylic acid (11.4 g, 90 mmol) in THF (500 ml) at 0° C., was added dropwise lithium aluminium hydride (solution 1M in THF, 117 m1, 117 mmol) and the mixture was stirred at room temperature overnight and then at 50° C. for 1 hour. Then water (3 ml) was added followed by Na₂SO₄, and the resulting precipitate was filtered off on a celite pad. The filtrate was concentrated under reduced pressure to afford the title compound as a solid (8 g, 78.95%); ¹H NMR (300 MHz, CDCl₃, ppm) δ: 7.25 (s, 1H), 6.7 (a, 1H), 5.25 (m, 1H), 4.4 (s, 2H), 3.45 (s, 3H).

### Intermediate 2: 1-methyl-4-chloromethyl-imidazole hydrochloride

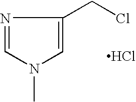

To a solution of intermediate 1 (5 g, 44.64 mmol) in CH₂Cl₂ (10 ml) at 0° C. was added dropwise thionyl chloride (50 ml) and then the mixture was stirred at room temperature overnight and then under reflux for 3 hours and then concentrated under reduced pressure. The residue was treated with diethyl oxide and the resulting precipitate was filtered and dried. The title compound was obtained as a brown solid (4 g, 53.81%); ¹H NMR (300 MHz, d⁶-DMSO, ppm) δ: 9.25 (s, 1H), 7.8 (s, 1H), 4.95 (s, 2H), 3.9 (s, 3H).

### Intermediate 3: 1-ethyl-4-(4-bromophenyl)-piperazine

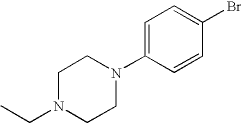

To a solution of 1-ethyl-4-phenyl-piperazine (18 g, 95 mmol) in ethanol (600 ml) cooled in an iced bath, was added dropwise bromine (5.1 ml, 99 mmol). The mixture was stirred at room temperature for 2 hours and then poured into water. The solution was made basic by addition of a solution of 1N sodium hydroxide. After extraction with CH₂Cl₂, the organic phase was dried over Na₂SO₄ and concentrated under reduced pressure. The residue was purified by chromatography on silica gel eluting with CH₂Cl₂/MeOH (9/1). The titled compound was obtained as a solid (21 g, 82.4%); [APCl MS] m/z 270 (MH⁺).

### Intermediate 4: 4-bromophenyl-morpholine

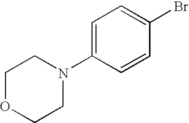

4-Phenyl-morpholine (18 g, 110.4 mmol) was reacted as described for intermediate 3 to afford, after crystallisation from diisopropyl oxide, the titled compound as a white solid (15 g, 56.13%); m p. 126-128° C.

### Intermediate 5: 4-(2-(pyrrolidin-1-yl)-ethoxy)-iodobenzene

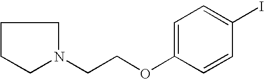

To a solution of 4-iodo-phenol (6 g, 27.3 mmol) in acetone (200 ml) were added cesium carbonate (22.2 g, 68.4 mmol) and N-(2-chloroethyl)-pyrrolidine hydrochloride (7 g, 41 mmol) and the mixture was heated under reflux during 4 hours and then poured into water. After extraction with CH₂Cl₂, the organic phase was dried over Na₂SO₄ and concentrated under reduced pressure. The titled compound was obtained as a red oil (8 g, 92.53%); ¹H NMR (300 MHz, CDCl₃, ppm) δ: 7.5 (d, 2H), 6.65 (d, 2H), 4 (t, 2H), 2.8 (t, 2H), 2.55 (m, 4H), 1.75 (m, 4H).

### Intermediate 6: 1-ethyl-4-[4-(4,4,5,5-tetramethyl-1,3,2dioxaborolan-2-yl)phenyl]-piperazine

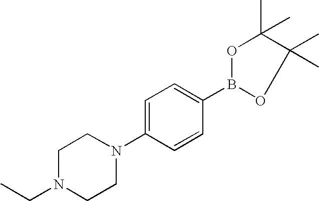

To a solution of intermediate 3 (3 g, 1 mmol) in dioxane (100ml) was added 4,4,5,5-tetramethyl-1,3,2dioxaborolane (1.8 ml, 12 mmol), dichlorobis(triphenylphosphine)palladium(II) (0.3929, 0.57 mmol), triethylamine (4.65 ml, 33 mmol) and the mixture was heated under reflux for 12 hours and then poured into water. After extraction with CH₂Cl₂, the organic phase was dried over Na₂SO₄ and concentrated under reduced pressure. The residue was purified by chromatography on silica gel eluting with CH₂Cl₂/MeOH (9/1). The titled compound was obtained as a brown oil which crystallised on standing (2 g, 55.48%); m.p. 130-134° C.

### Intermediate 7: 4-[4-(4,4,5,5-tetramethyl-1,3,2-dioxaborolan-2-yl)phenyl]-morpholine

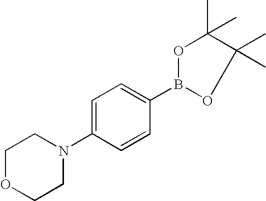

Intermediate 4 (15 g, 62 mmol) and 4,4,5,5-tetramethyl-1,3,2-dioxaborolane (9.8 ml, 68 mmol) were reacted as described for intermediate 6 to afford the titled compound as a solid (15 g, 83.74%); m.p. 114-116° C.

### Intermediate 8: 1-[2-(pyrrolidin-1-yl)-ethoxy]-4-[4,4,5,5-tetramethyl-1,3,2-dioxaborolan-2-yl]-benzene

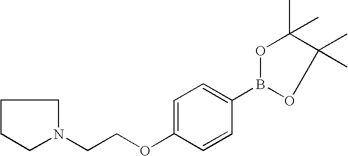

Intermediate 5 (8 g, 25.24 mmol) and 4,4,5,5-tetramethyl-1,3,2-dioxaborolane (4 ml, 27.6 mmol) were reacted as described for intermediate 6 to afford the titled compound as a solid (8 g, 99.99%); m.p. 160-164° C.

### Intermediate 9: 1-[aminocarbonylmethyloxy]-4-[4,4,5,5-tetramethyl-1,3,2-dioxaborolan-2-yl]-benzene

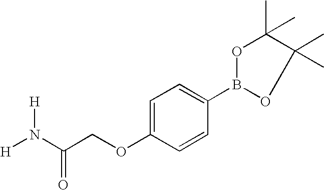

To a solution of 4-[4,4,5,5tetramethyl-1,3,2-dioxaborolan-2-yl]-phenol (5 g, 22.7 mmol) in acetone (80 ml) were added cesium carbonate (10.37 g, 32 mmol) and bromoacetamide (4.39 g, 32 mmol) and the mixture was heated at 70° C. for 3 hours and then concentrated under reduced pressure. The residue was treated with water and extracted with CH₂Cl₂. The organic phase was dried over Na₂SO₄, and concentrated. After trituration with diisopropyl oxide, the title compound was obtained as a solid (4 g, 63.54%); m.p. 166-168° C.

### Intermediate 10: 1-[(1-methyl-imidazol-4-yl)-methyloxy]-4-[4,4,5,5-tetramethyl-1,3,2-dioxaborolan-2-yl]-benzene

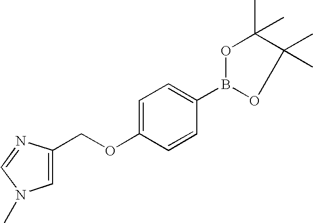

4-[4,4,5,5-Tetramethyl-1,3,2-dioxaborolan-2-yl]-phenol (1 g, 4.54 mmol) and intermediate 2 (1.88 g, 11.4 mmol) were reacted as described for intermediate 9 to afford, after chromatography on silica gel (CH₂Cl₂/MeOH, 95:5), the title compound as a pale yellow oil (0.5 g, 35%); ¹H NMR (300MHz, CDCl₃, ppm) δ: 7.6 (d, 2H), 7.3 (s, 1H), 6.8 (m, 3H), 4.9 (s, 2H), 3.5(s, 3H), 1.2 (s, 12H).

### Intermediate 11: 1-[(morpholin-4-yl)carbonylmethyloxy]-4-[4,4,5,5-tetramethyl-1,3,2-dioxaborolan-2-yl]-benzene

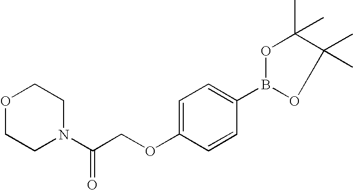

To a solution of 4-[4,4,5,-tetramethyl-1,3,2-dioxaborolan-2-yl]-phenol (6.6 g, 30 mmol) in CH₃CN were added potassium carbonate (12.42 g, 90 mmol) and N-(chloroacethyl)-morpholine (4.89 g, 30 mmol) and the mixture was heated under reflux for 3 hours and then concentrated under reduced pressure. The residue was treated with water and extracted with ethyl acetate. The organic phase was dried over Na₂SO₄, and concentrated. After trituration with hexane, the title compound was obtained as a grey solid (9.5 g, 91%); mnp. 112° C.; [APCl MS] m/z 348 (MH+).

### Intermediate 12: N-[(4-(4,4,5,5-tetramethyl-[1,3,2]dioxaborolan-2-yl)-phenyl)carbonyl]-morpholine

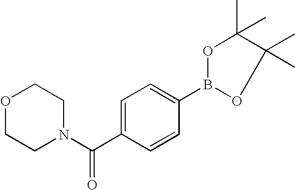

To d solution of 4-(4,4,5,5-tetramethyl-[1,3,2]dioxaborolan-2-yl)-benzoic acid (5 g, 20.15 mmol) in CH₂Cl₂/DMF (50 ml/5 ml) were added morpholine (2.1 ml, 24.2 mmol), HOBT (3.3 g, 24.2 mmol), EDCl (4.65 g, 24.2 mmol) and triethylamine (4.2 ml, 30.2 mmol) and the reaction mixture was stirred at room temperature for 3 days. Water was added and the product was extracted with CH₂Cl₂, the organic phase was dried over Na₂SO₄, and concentrated under reduced pressure. After trituration with diisopropyl oxide, the title compound was obtained as a white solid (4.21 g, 66%); ¹H NMR (300 MHz, CDCl₃, ppm) δ: 7.8 (d, 2H), 7.4 (d, 2H), 3.7 (m, 4H), 3.55 (m, 2H), 3.35 (m, 2H), 1.3 (s, 12H).

### Intermediate 13: 4-(4,4,5,5-tetramethyl-[1,3,2]dioxaborolan-2-yl)-N-(tetrahydro-pyran-4-yl)-benzamide

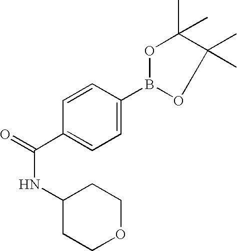

4-(4,4,5,5-Tetramethyl-[1,3,2]dioxaborolan-2-yl)-benzoic acid (70.16 g, 0.28 mol) was treated with SOCl₂ (2 vol.) and the reaction mixture was stirred to reflux for 2 hours. After evaporation, the residue was diluted in toluene and poured into a solution at 10° C. of tetrahydro-pyran-4-ylamine (34.34 g, 0.339) and triethylamine (79 mL, 0.57 mol) in CH₂Cl₂. The reaction mixture was stirred at room temperature for 2 days and water (490 mL) was added to give a precipitate which was filtered off and washed with ethyl acetate. After purification by flash chromatography using CH₂Cl₂/MeOH (95:5). The title compound was obtained as a solid (17.02 g, 18%); ¹H NMR (400 MHz, CDCl₃, ppm) δ: 7.85 (d, 2H), 7.72 (d, 2H), 5.98 (m, 1H), 4.20 (s, 1H), 3.99 (m, 2H), 3.35 (t, 2H), 2.01 (d, 2H), 1.57 (m, 2H), 1.35 (s, 12H).

### Intermediate 14: 1-ethyl-4-[((4,4,5,5-tetramethyl-[1,3,2]dioxaborolan-2-yl)-phenyl)carbonyl]-piperazine

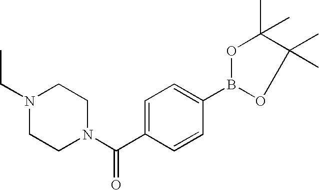

4-(4,4,5,5-Tetramethyl-[1,3,2]dioxaborolan-2-yl)-benzoic acid (8.24 g, 33.22 mmol) and N-ethylpiperazine (5.1 ml, 39.87 mmol) were reacted as described for intermediate 12 to afford, after chromatography on silica gel (CH₂Cl₂/MeOH, 95:5), the title compound as a pale yellow oil which crystallised (9.64 g, 84%); [APCl MS] m/z 345 (MH⁺).

### Intermediate 15: 2-(4-bromophenyl)-4-methyl-pyridine

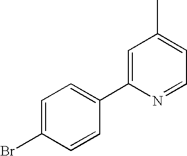

2-Brorno-4-methylpyridine (10 g, 58.14 mmol) was dissolved in toluene (100 ml) and tetrakis(triphenylphosphine)palladium(0) (5 mol %, 3.36 g) added under N₂ and degassed. Aqueous sodium carbonate (2M, 2 eq) was added slowly and stirred for 10 min. A solution of 4-bromophenylboronic acid (Lancaster, 14 g, 1.2 eq) in ethanol (20 ml) was added dropwise and the mixture was heated under reflux overnight and then poured into water. After extraction with CH₂Cl₂, the organic phase was dried over Na₂SO₄, and concentrated under reduced pressure. The resulting residue was purified by chromatography on silica gel (CH₂Cl₂/cyclohexane 6:4 then 8:2 then CH₂Cl₂). After crystallisation from pentane, the title compound was obtained as white crystals (6.3 g, 43.7%); ¹H NMR (300 MHz, CDC₃, ppm) δ: 8.5 (d, 1H), 7.83 (d, 2H), 7.56 (d, 2H), 7.5 (s, 1H), 7.05 (m, 1H), 2.4 (s, 3H).

### Intermediate 16: 2-[4(morpholin-4-yl)phenyl]-4-methyl-pyridine

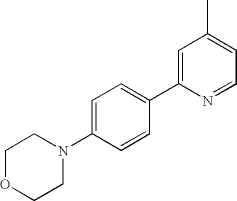

To a solution of intermediate 15 (2.66 g, 10.72 mmol) in toluene (50 ml) was added morpholine (1.12 ml, 1.2 eq, 12.9 mmol), Pd₂(dba)₃ (0.49 g, 0.05 eq, 0.53 mmol), binap (1 g, 0.15 eq, 1.6 mmol) and potassium tert-butoxide (1.44 g, 1.4 eq, 15 mmol) and the mixture was heated under reflux for 2 h and then poured into water. After extraction with CH₂Cl₂, the organic phase was dried over Na₂SO₄, and concentrated under reduced pressure. The resulting residue was purified by chromatography on silica gel (CH₂Cl₂/MeOH gradient from 99:1 to 95:5). The title compound was obtained as a yellow solid (2.6 g, 95.43%); ¹H NMR (300 MHz, CDCl₃, ppm) δ: 8.5 (d, 1H), 7.95 (d, 2H), 7.5 (s, 1H), 7 (m, 3H), 3.9 (m, 4H), 3.3 (m, 4H), 2.4 (s, 3H).

### Intermediate 17: 2-[2-(4-(morpholin-4-yl)phenyl)-pyridin-4-yl]-1-pyridin-2-yl-ethanone

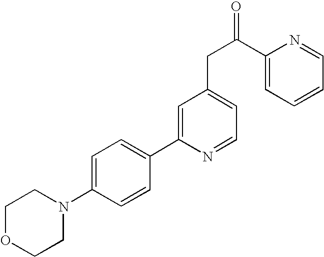

To a solution of Intermediate 16 (2.6 g, 10.24 mmol ) in dry THF (100 ml) under argon, was added dropwise a solution of sodium bis(trimethylsilyl)amide (1 M in THF, 22.52 ml, 2.2 eq, 22.53 mmol). The solution was stirred at room temperature for 0.5 h, then a solution of ethyl picolinate (1.66 ml, 1.2 eq, 12.3 mmol) in dry THF (20 ml) was added dropwise and the reaction mixture stirred at room temperature for 4 hours. The solvent was evaporated under reduced pressure and the solid precipitated with diisopropyl oxide. The brown solid was then taken up in saturated NH₄Cl solution and the aqueous phase extracted with CH₂Cl₂. The organic layer was dried over sodium sulfate and concentrated under reduced pressure to leave a residue which was purified by chromatography on silicagel (CH₂Cl₂ then CH₂Cl₂/MeOH gradient from 99:1 to 97:3). The title compound was obtained as an orange oil (1.42 g, 38.64%); ¹H NMR (300 MHz, CDCl₃, ppm) δ: 8.7 (d, 1H), 8.55 (d, 1H), 8.05 (d, 1H), 7.9 (d, 2H), 7.8 (m, 1H), 7.5 (m, 1H), 7.15 (m, 1H), 6.95 (m, 3H), 4.55 (s, 2H), 3.85 (m, 4H), 3.2 (m, 4H).

### Intermediate 18: 2-[2-bromo-pyridin-4-yl]-1-pyridin-2-yl-ethanone

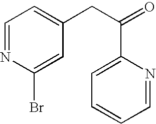

To a solution of 2-bromo-4-methyl-pyridine (27 g) in dry THF (270 ml) was added ethyl picolinate (28.5 g). The resulting mixture was cooled to −78° C. under argon and a solution of sodium bis(trimethylsilyl)amide (1M in THF, 345 ml) was added dropwise at −78° C. The resulting reaction mixture was allowed to reach room temperature and subsequently stirred overnight. The solvent was evaporated under reduced pressure and the solid residue triturated with Et₂O, filtered and washed with Et₂O. The solid was then diluted with saturated NH₄Cl solution and the aqueous phase extracted with ethyl acetate. The organic layer was dried over sodium sulfate and concentrated. The resulting orange powder was washed with pentane to give the title compound as a yellow solid (33.97 g); m.p. 111.2° C.

### Intermediate 19: 2-[2-Bromo-pyridin-4-yl]-1-(6-methyl-pyridin-2-yl)-ethanone

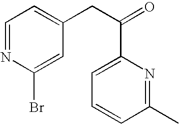

To a solution of 2-bromo-4-methyl-pyridine (5 g, 29 mmol) in dry THF (70 ml), a solution of sodium bis(trimethylsilyl)amide 2M in THF (32 ml, 2.2eq) was added dropwise at −30° C. under nitrogen. The mixture was stirred at −30° C. for 1 h, then 6-methylpicolinic acid methyl ester (4.82 g, 32.3 mmol, 1.1 eq) was added. The reaction mixture was stirred at room temperature overnight. Et₂O was added and the precipitated solid filtered and washed with Et₂O. The solid was then treated with saturated NH₄Cl solution and the aqueous phase extracted with EtOAc. The organic layer was dried over Na₂SO₄ and concentrated. The resulting orange powder was washed with pentane to give the title compound as a yellow solid (5.84 g, 70%); [APCl MS] m/z: 292 (MH⁺).

### Intermediate 20: 2-[2-(4-(methanesulfonyl)phenyl)-pyridin-4-yl]-1-(6-methylpyridin-2-yl)-ethanone

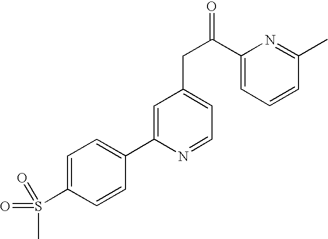

To a solution of intermediate 19 (2 g, 6.87 mmol) in, DME (80 ml) was added 4-(methanesulfonyl)-phenyl boronic acid (2.1 g, 10.31 mmol), tetrakis(triphenylphosphine)palladium(0) (0.4 g, 0.35 mmol), Na₂CO₃ (solution 2M, 22 ml) and the mixture was heated under reflux overnight and then poured into water. After extraction with CH₂Cl₂, the organic phase was dried over Na₂SO₄, and concentrated under reduced pressure. The residue was purified by chromatography on silica gel eluting with CH₂Cl₂/MeOH (95:5). The title compound was obtained as a yellow oil (1.1 g, 43.73%); [APCl MS] m/z: 367 (MH⁺).

### Intermediate 21: 2-[2-(4-(4-ethylpiperazin-1-yl)-phenyl)-pyridin-4-yl]-1-(6-methylpyridin-2-yl)-ethanone

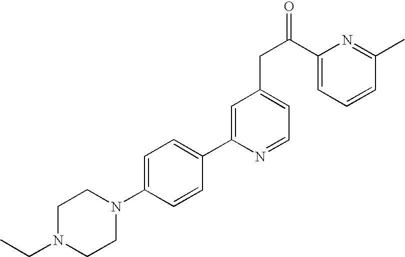

A mixture of intermediate 19 (0.6 g, 2 mmol) and intermediate 6 ( 0.76 g, 2.4 mmol) were reacted as described for intermediate 20 to afford the title compound as a yellow solid (0.5 g, 62.8%); [APCl MS] m/z 387 (MH⁺).

### Intermediate 22: 2-[2-(4-(morpholin-4-yl)-phenyl)-pyridin-4-yl]-1-(6-methylpyridin-2-yl)-ethanone

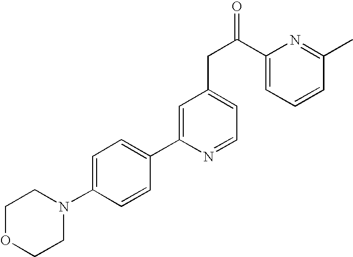

A mixture of intermediate 19 (1.4 g, 4.8 mmol) and intermediate 7 (1.8 g, 6.2 mmol) were reacted as described for intermediate 20 to afford the title compound as a yellow oil (1.2 g, 66.9%); [APCl MS] m/z 374 (MH⁺).

### Intermediate 23: 2-[2-(4-(methoxycarbonyl)-phenyl)-pyridin-4-yl]-1-(6-methylpyridin-2-yl)-ethanone

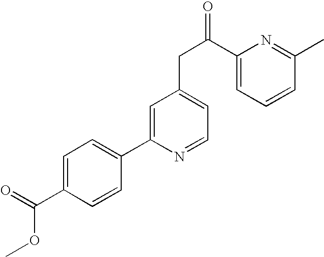

A mixture of intermediate 19 (2 g, 6.9 mmol) and 4-(methoxycarbonyl)-phenylboronic acid (1.9 g, 10.4 mmol) were reacted as described for intermediate 20 to afford the title compound as an orange oil which crystallised on standing (1.2 g, 50.5%); [APCl MS] m/z 347 (MH⁺).

### Intermediate 24: 2-[2-(4-(formyl)-phenyl)-pyridin-4-yl]-1-(6-methylpyridin-2-yl)-ethanone

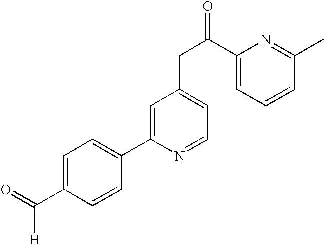

A mixture of intermediate 19 (1 g, 3.4 mmol) and 4-(formyl)-phenylboronic acid (0.78 g, 5.16 mmol) were reacted as described for intermediate 20 to afford the title compound as an orange oil (1 g, 92.1%); [APCl MS] m/z 317 (MH⁺).

### Intermediate 25: 2-[2-(4-methoxyphenyl)-pyridin-4-yl]-1-(6-methylpyridin-2-yl)-ethanone

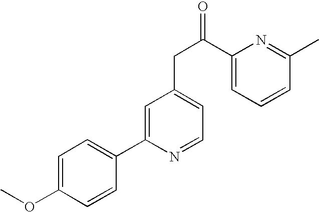

A mixture of intermediate 19 (1 g, 3.4 mmol) and 4-methoxyphenylboronic acid (0.63 g, 4 mmol) were reacted as described for intermediate 20 to afford the title compound as a yellow oil (0.3 g, 27.45%); [APCl MS] m/z 319 (MH⁺).

### Intermediate 26: 2-[2-(4-trifluoromethoxyphenyl)-pyridin-4-yl]-1-(6-methylpyridin-2-yl)-ethanone

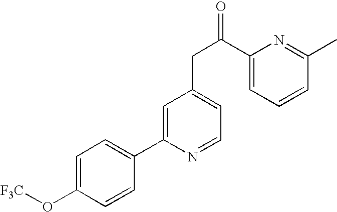

A mixture of intermediate 19 (2 g, 7 mmol) and 4-trifluoromethoxyphenylboronic acid (1.55 g, 7.7 mmol) were reacted as described for intermediate 20 to afford the title compound as a yellow powder (1.6 g, 62.58%); m.p. 76-78° C.

### Intermediate 27: 2-[2-(4-(2-(pyrolidin-1-yl)ethoxy)phenyl)-pyridin-4-yl]-1-(6-methylpyridin-2-yl)-ethanone

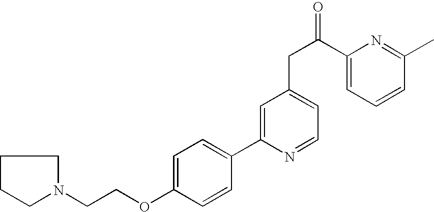

A mixture of intermediate 19 (1 g, 3.4 mmol) and intermediate 8 (1.2 g, 3.8 mmol) were reacted as described for intermediate 20 to afford the title compound as a yellow oil (0.3 g, 21.8%); [APCl MS] m/z 402 (MH⁺).

### Intermediate 28: 2-[2-(4-(aminocarbonylmethyloxy)phenyl)-pyridin-4-yl]-1-(6-methylpyridin-2-yl)-ethanone

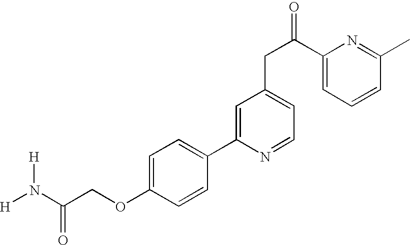

A mixture of intermediate 19 (2 g, 6.9 mmol) and intermediate 9(2.1 g, 7.6 mmol) were reacted as described for intermediate 2O to afford the title compound as a brown powder (1.2 g, 48.37%); m.p. 144-146° C.

### Intermediate 29: 2-[2-(4-(1-methyl-imidazol-4-yl)-phenyl)-pyridin-4-yl]-1-(6-methylpyridin-2-yl)ethanone

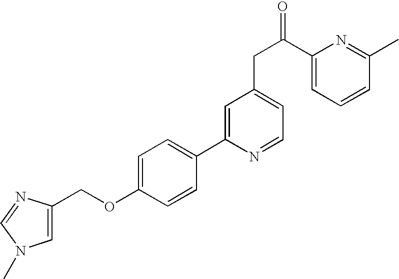

A mixture of intermediate 19 (0.357 g, 1.22 mmol) and intermediate 10 (0.5 g, 1.59 mmol) were reacted as described for intermediate 20 to afford the title compound as a yellow oil (0.15 g, 30%); [APCl MS] m/z 399 (MH⁺).

### Intermediate 30: 2-[2-(4-(carboxy)-phenyl)-pyridin-4-yl]-1-(6-methylpyridin-2-yl)-ethanone

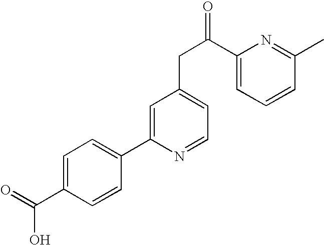

To a solution of intermediate 23 (1.2 g, 3.47 mmol) in MeOH (100 ml) was added sodium hydroxide (solution 1N, 5 ml, 5.2 mmol) and the mixture was heated under reflux for 48 hours. After cooling, a solution of 1N HCl (5 ml) was added and the precipitate was filtered and dried. The titled compound was obtained as an orange solid (0.8 g, 69.5%); [APGI MS] m/z 333 (MH⁺).

### Intermediate 31: 2-[2-(4-((morpholin-4-yl)carbonyl)-phenyl)-pyridin-4-yl]-1-(6-methylpyridin-2-yl)-ethanone

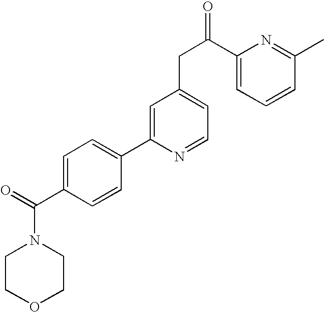

To a solution of intermediate 30 (0.8 g, 2.41 mmol) in CH₂Cl₂ (50 ml) were added morpholine (0.32 ml, 3.61 mmol), HOBT (0.49 g, 3.61 mmol), EDCl (0.63 g, 3.61 mmol), triethylamine (0.84 ml, 6 mmol) and the mixture was stirred at room temperature for 24 hours and then poured into water. After extraction with CH₂Cl₂, the organic phase was dried over Na₂SO₄, and concentrated under reduced pressure. The residue was purified by chromatography on silica gel eluting with CH₂Cl₂/MeOH (90:10). The title compound was obtained as an orange oil (0.8 g, 85.35%); [APCl MS] m/z 390 (MH⁺).

### Intermediate 32: 2-{2-[4-((tetrahydropyran-4-yl)-aminocarbonyl)-phenyl]pyridin-4-yl}-1-(6-methylpyridin-2-yl)-ethanone

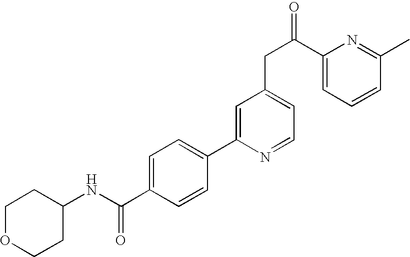

Intermediate 30 (1 g, 3 mmol) and 4-amino-tetrahydropyran (340 mg, 3.3 mmol) were reacted as described for intermediate 31 to afford, after chromatography on silicagel (CH₂Cl₂/MeOH, 90:10), the title compound as a yellow oil (0.3 g, 24%); ¹H NMR (300 MHz, CDCl₃, ppm) δ: 8.58 (d, 1H), 7.98 (m, 2H), 7.8 (m, 3H), 7.66 (m, 2H), 7.29 (m, 1H), 7.19 (m, 1H), 6.01 (m, 1H), 4.57 (s, 2H), 4.15 (m, 1H), 3.93 (m, 2H), 3.48 (m, 2H), 2.6 (s, 3H), 1.95 (m, 2H), 1.55 (m, 2H).

### Intermediate 33: 2-[2-(4-((morpholin-4-yl)methyl)-phenyl)-pyridin-4-yl]-1l-(6-methylpyridin-2-yl)-ethanone

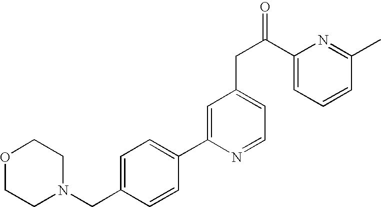

To a solution of intermediate 24 (1 g, 3.2 mmol) in CH₂Cl₂ (100 ml) were added morpholine (0.36 g, 4.1 mmol) and sodium triacetoxyborohydride (0.88 g, 4.1 mmol) and the mixture was stirred at room temperature for 3 hours and then poured into a saturated solution of NaHCO₃. After extraction with CH₂Cl₂, the organic phase was dried over Na₂SO₄, and concentrated under reduced pressure. The titled compound was obtained as a yellow oil (1.1 g, 89.82%); [APCl MS] m/z 388 (MH⁺).

### Intermediate 34: [(2-(4-ethanesulfonylphenyl)-Pyridin-4-yl)-(Phenylamino)-methyl]-phosphonic acid diphenylester

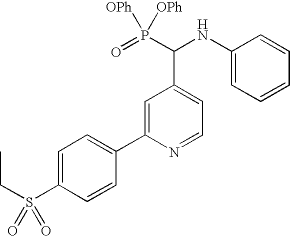

To a solution of 2-chloro-pyridine-4-carboxaldehyde (1 g, 7.06 mmol) in DME (50 ml) was added 4-(ethanesulfonyl)-phenyl boronic acid (1.97 g, 9.18 mmol), tetrakis(triphenylphosphine)palladium(0) (0.816 g, 0.7 mmol), Na₂CO₃ (solution 2M, 7 ml) and the mixture was heated under reflux overnight and then poured into water. After extraction with CH₂Cl₂, the organic phase was dried over Na₂SO₄, and concentrated under reduced pressure. The residue was purified by chromatography on silica gel eluting with CH₂Cl₂/MeOH (99/1) to afford 2-(4-ethanesulfonylphenyl)-pyridine-4-carboxaldehyde as a yellow oil (1.94 g, 98%). To a solution of 2-(4-ethanesulfonylphenyl)-pyridine-4-carboxaldehyde (1.94 g, 7.06 mmol) in iPrOH were added aniline (0 772 ml, 8.47 mmol) and diphenylphosphite (1.91 ml, 9.9 mmol) and the mixture was stirred at room temperature for 18 hours and then concentrated under reduced pressure. The residue was treated with water and extracted with CH₂Cl₂, the organic phase was dried over Na₂SO₄ and concentrated. After chromatography on silicagel (CH₂Cl₂), the title compound was obtained as a yellow oil (1.45 g, 35.13%); [APCl MS] m/z 585 (MH⁺).

### Intermediate 35: 1-[2-(4-ethanesulfonylphenyl)-pyridin-4-yl]-2-[6-methyl-pyridin-2-yl]-ethanone

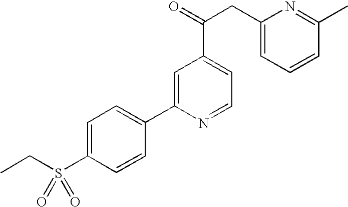

To a solution of intermediate 34 (1.45 g, 2.48 mmol) in THF/iPrOH were added 6-methyl-pyridine-2-carboxaldehyde (0.251 g, 2.07 mmol) and cesium carbonate (1.35 g, 4.14 mmol) and the mixture was stirred at room temperature for 18 hours and then neutralised with a solution of sodium bicarbonate. After concentration under reduced pressure, the residue was treated with water and extracted with CH₂Cl₂. The organic phase was dried over Na₂SO₄ and concentrated under reduced pressure. After chromatography on silica gel (CH₂Cl₂/MeOH, 99:1), the title compound was obtained as a yellow oil (0.321 g, 34.02%); [APCl MS] m/z 381 (MH⁺).

### Intermediate 36: 2-bromo-N-methoxy-N-methyl-4-pyridinecarboxamide

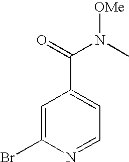

To a suspension of 2-bromo-4-pyridinecarboxylic acid (23.5 g, 116 mmol) in CH₂Cl₂ (600 mL) were added under nitrogen HOBT (17.3 g, 128 mmol), EDCl (24.5 g, 128 mmol), triethylamine (46.85 g, 464 mmol) and N,O-dimethylhydroxylamine hydrochloride (17.02 g, 175 mmol). The reaction mixture was stirred at room temperature for 3 h and then partitioned between water and CH₂Cl₂. The organic phase was dried over Na₂SO₄, filtered and evaporated under reduced pressure to afford the title compound as a white solid (17 g, 59.64%); [APCl MS] m/z 246 (MH+).

### Intermediate 37: 1-[2-bromo-pyridin-4-yl]-2-[6-methyl-pyridin-2-yl]-ethanone

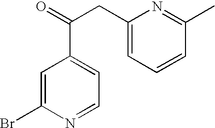

2,6-Lutidine (4.28 g; 40 mmol) was dissolved in dry THF (100 mL) under nitrogen and the solution was cooled to −30° C. 2.5M n-Butyllithium in hexanes (16 mL; 40 mmol) was added at −30° C., then the mixture was stirred 1.5 h at ambient temperature before being cooled to −30 to −40° C. A solution of intermediate 36 (4.9 g, 20 mmol) in dry THF (20 mL) was added at −40° C. and the reaction stirred for 2 h. Saturated aqueous ammonium chloride was added and the mixture was extracted with EtOAc. The organic phase was dried over Na₂SO₄, filtered and evaporated under reduced pressure. The residue was purified by chromatography on silica gel (CH₂Cl₂/MeOH, 99/1) to give the title compound (3.42 g; 58%) as a yellow solid; m.p. 126° C.; [APCl MS] m/z 292(MH⁺).

### Intermediate 38: 2-(6-methylpyridin-2-yl)-1-[2-(4-chlorophenyl)-pyridin-4]-ethanone

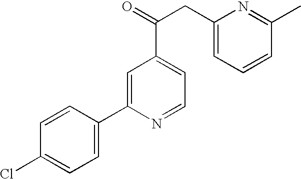

To a solution of intermediate 37 (0.582 g, 2 mmol) in a mixture DME (30ml)/water (15 ml) were added 4-chloro-phenyl boronic acid (0.405 g, 2.6 mmol), tetrakis(triphenylphosphine) palladium(0) (0.06 g, mmol) and sodium carbonate solution (2M, 4 ml). The reaction mixture was heated for 30 min and then poured into water. After extraction with CH₂Cl₂, the organic phase was dried over Na₂SO₄, and concentrated under reduced pressure to afford the title compound as a solid (0.415 g, 64.44%); m.p. 94° C.; [APCl MS] m/z 323 (MH⁺).

### Intermediate 39: 2-(6-methylpyridin-2-yl)-1-[2-(4-trifluoromethoxyphenyl)-pyridin-4-yl]-ethanone

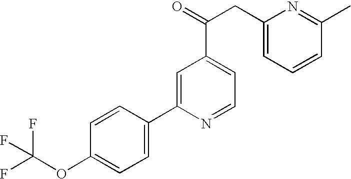

Intermediate 37 (0.582 g, 2 mmol) and 4-trifluoromethoxy-phenyl boronic acid (0.533 g, 2.6 mmol) were reacted as was described for intermediate 38 to give the title compound as a solid (0.72 g, 96.77%); m.p. 60° C.; [APCl MS] m/z 373 (MH⁺).

### Intermediate 40: 2-(6-methylpyridin-2-yl)-1-[2-(4-((morpholin-4-yl)carbonyl)phenyl)-pyridin-4-yl]-ethanone

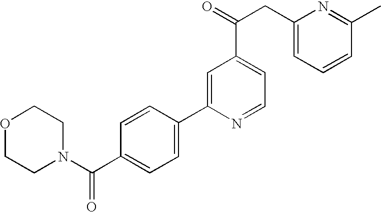

Intermediate 37 (1 g, 3.43 mmol) and intermediate 12 (1.2 g, 3.78 mmol), were reacted as was described for intermediate 38 to give the title compound as a yellow solid (1.25 g, 90.71%); m.p. 106° C.; [APCl MS] m/z 402 (MH⁺).

### Intermediate 41: 2-(6-methylpyridin-2-yl)-1-[2-(4-((1-ethyl-piperazin-4-yl)carbonyl)phenyl)-pyridin-4-yl]-ethanone

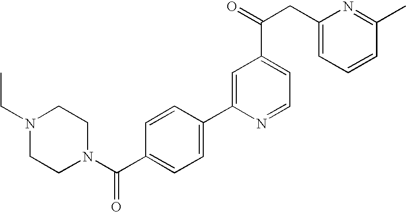

Intermediate 37 (1 g, 3.43 mmol) and intermediate 14 (1.28 g, 3.78. mmol) were reacted as was described for intermediate 38 to give the title compound as a yellow solid (0.95 g, 64.59%); m.p. 90° C.; [MS APCl] m/z 429 (MH⁺).

### Intermediate 42: 2-(6-methylpyridin-2-yl)-1-[2-(4-((tetrahydropyran-4-yl)-aminocarbonyl)phenyl)-pyridin-4-yl]-ethanone

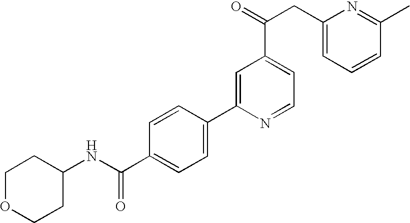

Intermediate 37 (1 g, 3.43 mmol) and intermediate 13 (1.25 g, 3.78 mmol) were reacted as was described for intermediate 38 to give the title compound as a yellow solid (1.4 g, 98.17%); m.p. 210° C.; [APCl MS] m/z 416 (MH⁺).

### Intermediate 43: 2-(6-methylpyridin-2-yl)-1-[2-(4-(morpholin-4-yl)phenyl)-pyridin-4-yl]-ethanone

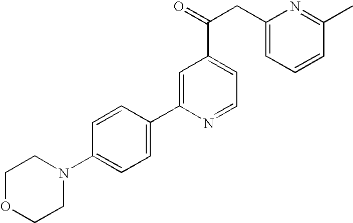

Intermediate 37 (1 g , 3.43 mmol) and intermediate 7 were reacted as was described for intermediate 38 to give the title compound as a yellow solid (1.2 g, 84%); m.p. 114° C.; [APCl MS] m/z 374 (MH⁺).

### Intermediate 44: 2-(6-methylpyridin-2-yl)-1-[2-(4-(2-(pyrolidin-1-yl)-ethoxy)-phenyl)-pyridin-4-yl]-ethanone

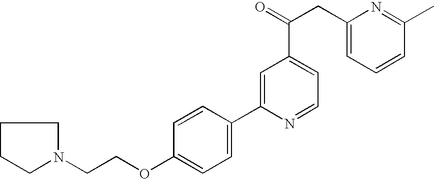

Intermediate 37 (1 g, 3.43 mmol) and intermediate 8 (1.2 g, 3.78 mmol) were reacted as was described for intermediate 38 to give, after chromatography on silica gel (CH₂Cl₂/MeOH, 90:10), the title compound as a yellow oil (0.8 g, 58%); [APCl MS] m/z 402 (MH⁺).

### Intermediate 45: 2-(6-methylpyridin-2-yl)-1-[2-(4-(aminocarbonylmethyloxy)-phenyl)-pyridin-4-yl]-ethanone

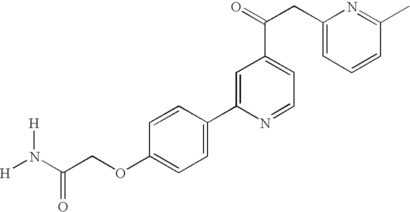

Intermediate 37 (1 g, 3.43 mmol) and intermediate 9 (0.93 g, 4 12 mmol) were reacted as was described for intermediate 38 to give the title compound as a yellow solid (1.2 g, 96%); m.p. 168° C.; [APCl MS] m/z 362 (MH⁺).

### Intermediate 46: 2-(6-methylpyridin-2-yl)-1-[2-(4-((morpholin-4-yl)-carbonylmethyloxy)-phenyl)-pyridin-4-yl]-ethanone

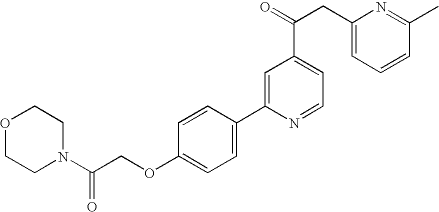

Intermediate 37 (1 g, 3.43 mmol) and intermediate 11 (1.45 g, 4.12 mmol) were reacted as was described for intermediate 38 to give, after chromatography on silica gel (CH₂Cl₂/MeOH, 98:2), the title compound as a yellow gum (1 g, 67.52%); [APCl MS] m/z 432 (MH⁺).

### Intermediate 47: 2-(6-methylpyridin-2-yl)-1-[2-(4-formylphenyl)-pyridin-4-yl]-ethanone

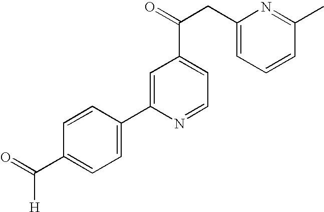

Intermediate 37 (2 g, 7 mmol) and 4-formylphenylboronic acid (1.34 g, 9 mmol) were reacted as was described for intermediate 38 to give the title compound as a yellow solid (2.1 g, 96.69%); m.p. 118° C.; [APCl MS] m/z 317 (MH⁺).

### Intermediate 48: 2-(6-methylpyridin-2-yl)-1-[2-(4-((morpholin-4-yl)methyl)-phenyl)-pyridin-4-yl]-ethanone

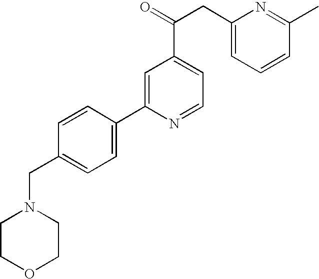

To a solution of intermediate 47 (0.984 g, 3 mmol) in 1,2-dichloroethane (40 ml) were added morpholine (0.34 g, 3.9 mmol), sodium triacetoxyborohydride (0.826 g, 3.9 mmol) and acetic acid (0.216 g, 3.6 mmol) and the mixture was stirred at room temperature for 3 hours and then poured into water. After extraction with CH₂Cl₂, the organic phase was dried over Na₂SO₄ and concentrated under reduced pressure. The title compound was obtained as an oil (1.1 g, 91%); [APCl MS] m/z 388 (MH⁺).

### Intermediate 49: 2-(6-methylpyridin-2-yl)-1-[2-(4-((pyrolidin-1-yl)methyl)-phenyl)-pyridin-4-yl]-ethanone

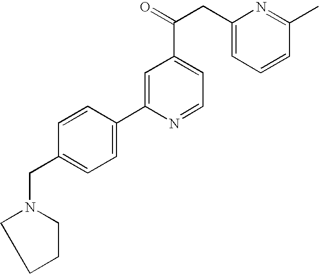

Intermediate 47 (0.7 g, 2.2 mmol) and pyrolidine (0.203 g, 2.8 mmol) were reacted as described for intermediate 48, to afford after chromatography on silica gel (CH₂Cl₂/MeOH, 90:10), the title compound as a yellow gum (0.5 g, 60.84%); [APCl MS] m/z 372 (MH⁺).

### Intermediate 50: 2-(6-methylpyridin-2-yl)-1-[2-(4-((dimethylamino)methyl)phenyl)-pyridin-4-yl]-ethanone

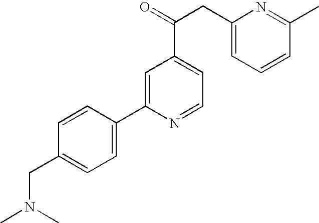

Intermediate 47 (0.7 g, 2.2 mmol) and dimethylamine (solution 2M in THF, 1.4 ml, 2.86 mmol) were reacted as described for intermediate 48, to afford after chromatography on silica gel (CH₂Cl₂/MeOH, 9/1), the title compound as a yellow gum (0.4 g, 52.34%); (APCl MS] m/z 346 (MH⁺).

### Intermediate 51: 1-[2-bromo-pyridin-4-yl]-2-[pyridin-2-yl]-ethanone

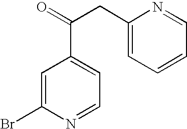

To a solution of 6-methylpyridine (2.79 g; 30 mmol) in dry THF (20 ml) under nitrogen cooled at −80° C., was added dropwise NaHMDS (solution 1M/THF, 36 ml, 36 mmol). and the mixture was stirred for 1 hour at −80° C. A solution of intermediate 36 (7.35 g; 30 mmol) in dry THF (10 mL) was added dropwise and the mixture was then stirred at room temperature overnight and then concentrated under reduced pressure. The residue was treated with hexane and the resulting precipitate was filtered. The solid was then diluted with saturated ammonium chloride solution and the aqueous phase extracted with ethyl acetate. The organic layer was dried over sodium sulfate and concentrated. After chromatography on silica gel (CH₂Cl₂/MeOH, 98:2), the title compound was obtained as a yellow solid (4.1 g, 49.34%); m.p. 96° C.

### Intermediate 52: 2-(pyridin-2-yl)-1-[2-(4-((tetrahydropyran-4-yl)aminocarbonyl)-phenyl)-pyridin-4-yl]-ethanone

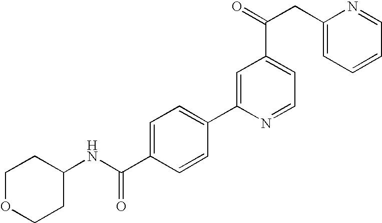

Intermediate 51 (0.95 g, 3.43 mmol) and intermediate 13 (1.25 g, 3.78 mmol) were reacted as described for intermediate 38 to afford, after chromatography on silica gel (CH₂Cl₂/MeOH, 97:3), the title compound as a yellow solid (0.8 g, 58.17%); [APCl MS] m/z 402 (MH⁺).

### Intermediate 53: 2-(pyridin-2-yl)-1-[2-(4-(morpholin-4-yl)-phenyl)-pyridin-4-yl]-ethanone

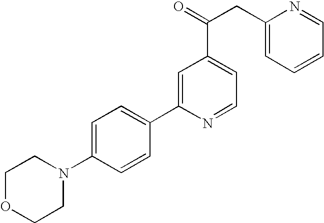

Intermediate 51 (0.95 g, 3.43 mmol) and intermediate 7 (1.1 1 g, 3.78 mmol) were reacted as described for intermediate 38 to afford, after chromatography on silica gel (CH₂Cl₂/MeOH, 96:4), the title compound as a yellow solid (0.6 g, 48.73%); [APCl MS] m/z 360 (MH⁺).

### Intermediate 54: Solid supported 5-(2-bromo-4-pyridinyl)-4-(2-pyridinyl)-1,3-thiazol-2-amine

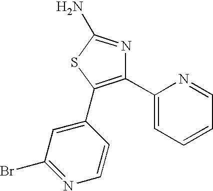

Step 1: Rink Argopore resin (12 g, 0.58 mmol/g substitution) was placed into a peptide vessel and washed with CH₂Cl₂ (3×100 mL). The resin was then treated for 10 min with a solution of piperidine 20% in DMF (3×40 mL). After washing with DMF (3×100 mL) and CH₂Cl₂ (3×100 mL), the resin was treated with a solution of Fmoc-NCS (0.2M) in CH₂Cl₂ (170 mL) under argon at room temperature for 1 h. The resin was washed with DMF (3×100 mL), EtOH (3×100 mL) and CH₂Cl₂ (3×100 mL) and subsequently stirred for 10 min with a solution of piperidine 20% in DMF (3×40 mL) to give after washing with DMF (3×100 mL) and CH₂Cl₂ (3×100 mL) the resin bound thiourea.

Step 2: To a solution of intermediate 18 (8.5 g, 29 mmol) in dioxane (145 mL) was added under argon polymer-supported pyridinium perbromide (1.8 mmol/g, 16 g). The suspension was shaken under argon at room temperature overnight. The resin was removed by filtration and washed with dioxane (25 mL) to give 2-bromo-2-(2-bromo-4-pyridinyl)-1-(2-pyridinyl)ethanone which was used in solution in dioxane without purification in the next step.

Step 3: The product from step 1 was stirred with 2-bromo-2-(2-bromo-4-pyridinyl)-1-(2-pyridinyl)ethanone (0.18M) in dioxane (175 mL) for 4 h at room temperature under argon. The resin was washed with dioxane (3×100 mL). A second exposure with 2-bromo-2-(2-bromo-4-pyridinyl)-1-(2-pyridinyl)ethanone (0.18M in dioxane, 175 mL) was performed. The resin was washed with DMF (3×100 mL), EtOH (3×100 mL), CH₂Cl₂ (3×100 mL) and dried under a stream of nitrogen overnight. 2 mg of the obtained resin were cleaved with a solution of TFA 20% in CH₂Cl₂ to give the title compound which was characterised by LC-MS (purity>96%); [APCl MS] m/z 333, 335, 336 (MH⁺).

### Intermediate 55: Solid supported 5-(2-bromo-4-pyridinyl)-4-(6-methyl-2-pyridinyl)-1,3-thiazol-2-amine

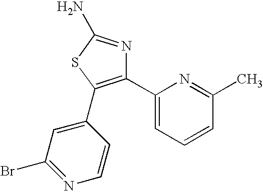

Intermediate 55 was prepared in analogous fashion to intermediate 54 starting from intermediate 19. After step 3, 2 mg of the obtained resin were cleaved with a solution of TFA 20% in CH₂Cl₂ to give the title compound which was characterised by LC-MS (purity>96%); [APCl MS] m/z 347/349/350 (MH+).

### EXAMPLES

### Example 1

### 5-{2-[4-morpholin-4)phenyl]pyridin-4-yl}-4-pyridin-2-yl)-1,3thiazol-2-amine

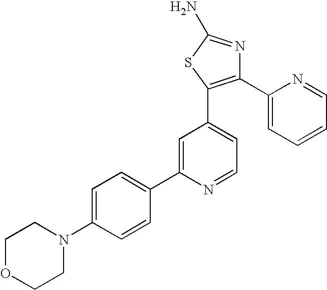

To a solution of Intermediate 17 (0.4 g, 1.11 mmol) in CH₂Cl₂ (20 ml) was added polymer-supported pyridinium perbromide (Fluka, 0.62 g, 1 eq, 1.11 mmol) and the suspension shaken for 50 min. The resin was removed by filtration, with the filtrate being added directly to thiourea (0.25 g, 3 eq, 3.33 mmol) and the resin washed several times with ethanol. The filtrate was heated at reflux overnight, allowed to cool at room temperature and concentrated. The residue was basified with aqueous NaOH, extracted into CH₂Cl₂. The organic phase was washed with water, dried over Na₂SO₄, and concentrated under reduced pressure. After chromatography on silica gel (CH₂Cl₂/MeOH, 95:5 then 90:10) and crystallisation from ethyl acetate, the title compound was obtained as cream crystals (108 mg, 23.35%); m.p. 246° C.; [APCl MS] m/z 416 MH⁺.

### Example 2

### 5-{2-[4-(methanesulfonyl)phenyl]pyridin-4-yl}-4-(6-methylpyridin-2-yl)-1,3-thiazol-2-amine

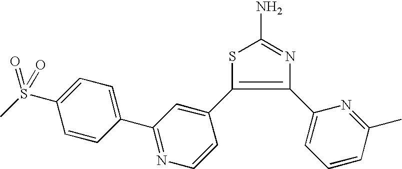

To a solution of Intermediate 20 (1.1 g, 3 mmol) in CH₂Cl₂ (100 ml) was added polymer-supported pyridinium perbromide (3 g) and the suspension was shaken for 3 hours. The resin was removed by filtration, with the filtrate being added directly to thiourea (0.3 g, 3.9 mmol) and the resin washed many times with ethanol. The filtrate was heated under reflux for 4 hours, allowed to cool and concentrated. The residue was basified with aqueous NaOH, extracted into CH₂Cl₂ and this phase washed with water. The organic phase was dried over Na₂SO₄ and concentrated under reduced pressure. After chromatography on silica gel (CH₂Cl₂/MeOH, 90:10) and trituration with pentane, the title compound was obtained as a solid (0.9 g, 71%); m.p. 236-238° C.; TOF MS ES⁺ exact mass calculated for C₂₁H₁₈N₄O₂S₂: calculated 423.0949 (MH⁺). Found 423.0945(MH⁺).

### Example 3

### 5-{2-[4-(4-ethylpiperazin-1-yl)phenyl]pyridin-4-yl-4-(6-methylpyridin-2-yl)-1,3-thiazol-2-amine

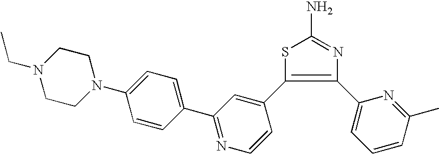

Intermediate 21 (0.5 g, 1.3 mmol) was reacted as described for example 1, to afford after crystallisation from EtOH the title compound as crystals (0.16 g, 27.1%); m.p 230-232° C.; TOF MS ES⁺ exact mass calculated for C₂₆H₂₈N₆S: calculated 457.2174 (MH⁺). Found 457.2213 (MH⁺)

### Example4

### 5-{2-[4-(morpholin-4-yl)phenyl]pyridinyl-4-yl}-4-(6-methylpyridin-2-yl)-1,3-thiazol-2-amine

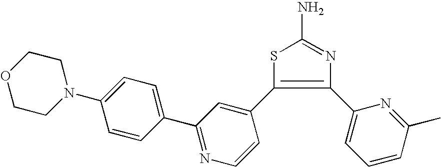

Intermediate 22 (1.2 g, 3.2 mmol) was reacted as described for example 1, to afford after crystallisation from MeOH the title compound as cream crystals (400mg, 28.98%); m.p. 250-252° C.; TOF MS ES⁺ exact mass calculated for C₂₄H₂₃N₅OS calculated 430.1701 (MH⁺). Found 430.1698 (MH⁺).

### Example 5

### 5-{2-[4-((morpholin-4-yl)carbonyl)phenyl]pyridin-4-yl]-4-(6-methylpyridin-2-y)-1,3-thiazol-2-amine

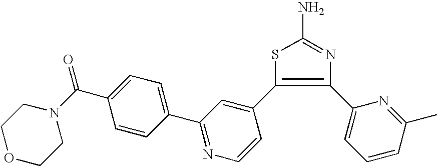

Intermediate 31 (0.8 g, 2 mmol) was reacted as described for example 1 to afford, after crystallisation from acetonitrile the titled compound as cream crystals. (300 mg, 32%); m.p. 158-160° C.; [LC Tof] C₂₅H₂₃N₅O₂S₁ (MH⁺) calculated 458.1651 (MH⁺) found 458.1602; −4;9ppm.

### Example 6

### 5-{2-[4((tetrahydropyran-4-yl)-aminocarbonyl)-phenyl]pyridin-4-yl}-4-(6-methylpyridin-2-yl)-1,3-thiazol-2-amine

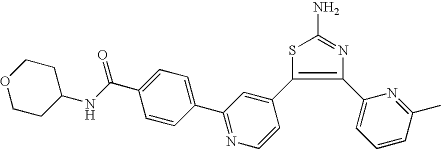

Intermediate 32 (0.3 g, 0.72 mmol) was reacted as described for example 1 to afford after trituration with pentane, the title compound as cream crystals (0.14 g, 41.12%); m.p. 230-232° C.; [APCl MS] m/z 472 (MH⁺).

### Example 7

### 5-{2-[4-((morpholin-4-yl)methyl)phenyl]pyridin-4-yl}-4-(6-methylpyridin-2-yl)-1,3-thiazol-2-amine

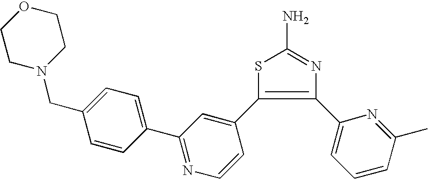

Intermediate 33 (1.1 g, 2.54 mmol) was reacted as described for example 1, to afford after crystallisation from EtOH the title compound as cream crystals (0.2 g, 15.9%); m.p. 190-192° C.; TOF MS ES⁺ exact mass calculated for C₂₅H₂₅N₅OS: 443.1860 (MH⁺).Found 443.1800 (MH⁺).

### Example 8

### 5-{2-[4methoxyphenyl]pyridin-4-yl}-4-(6methylpyridin-2-yl)-1,3-thiazol-2-amine

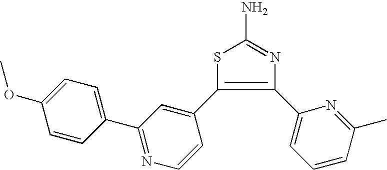

Intermediate 25 (0.3 g, 0.94 mmol) was reacted as described for example 1, to afford after crystallisation from acetonitrile the title compound as cream crystals (0.11 g, 31.18%); m.p. 188-190° C.; [APCl MS] m/z 375 (MH⁺).

### Example 9

### 5-{2-[4-trifluoromethoxyphenyl]pyridin-4-yl}-4-(6-methylpyridin-2-yl)-1,3-thiazol-2-amine

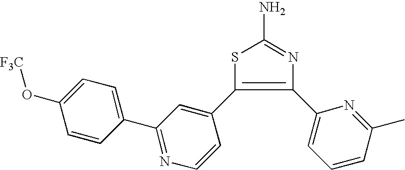

Intermediate 26 (1.6 g, 4.3 mmol) was reacted as described for example 1 to afford, after trituration with diisopropyl oxide, the title compound as a white solid (1.2 g, 65.19%); m.p. 222-224° C.; [APCl MS] m/z 429 (MH⁺).

### Example 10

### 5-{2-[4-(aminocarbonylmethyloxy)-phenyl]pyridin-4-yl}-4-(6-methylpyridin-2-yl)-1,3-thiazol-2-amine

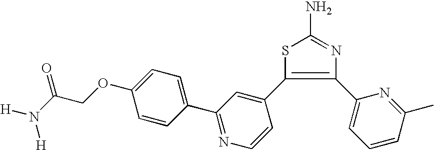

Intermediate 28 (1.2 g, 3.3 mmol) was reacted as described for example 1 to afford, after crystallisation from acetonitrile, the title compound as yellow crystals (0.2 g, 14.43%); m.p. 52-154° C.; [APCl MS] m/z 418 (MH⁺).

### Example 11

### 5-{2-[4-(2-(pyrrolidin-1-yl)-ethoxy)-phenyl]pyridin-4-yl}-4-(6-methylpyridin-2-yl)-1,3-thiazol-2-amine

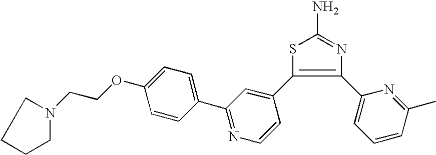

Intermediate 27 (0.3 g, 0.75 mmol) was reacted as described for example 1 to afford, after trituration with pentane, the title compound as cream crystals (0.11 g, 32.17%); m.p. 176-178° C.; [APCl MS] m/z 458 (MH⁺).

### Example 12

### 5-{2-[4-((1-methyl-imidazol-4-yl)methyloxy)-phenyl]pyridin-4-yl}-4-(6-methylpyridin-2-yl)-1,3-thiazol-2-amine

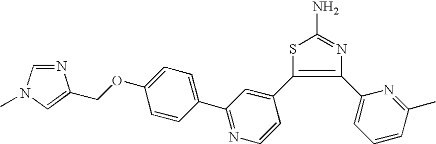

Intermediate 29 (0.15 g, 0.37 mmol) was reacted as described for example 1 to afford, after trituration with diisopropyl oxide, the title compound as a yellow solid (0.03 mg, 17.5%); m.p. 226-228° C.; TOF MS ES⁺ exact mass calculated for C₂₅H₂₂N₆OS: 455.1654 (MH⁺). Found 454.1600(MH⁺).

### Example 13

### 4-[2-(4-chlorophenyl)pyridin-4-yl]-5-(6-methylpyridin-2-yl]-1,3-thiazol-2-amine

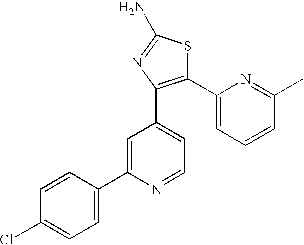

To a solution of Intermediate 38 (0.322 g, 1 mmol) in THF (10 ml) was added polymer-supported pyridinium perbromide (0.66 g, 1 mmol) and the suspension was shaken for 3 hours at room temperature. The resin was removed by filtration, with the filtrate being added directly to thiourea (0.152 g, 2 mmol) and the resin washed many times with ethanol. The filtrate was heated under reflux for 3 hours, allowed to cool and concentrated. Water was added to the residue and the resulting precipitate was filtered and dried. After crystallisation from EtOH, the title compound was obtained as white crystals (0.154 g, 40.74%); m.p. 222° C.; TOF MS ES⁺ exact mass calculated for C₂₀H₁₅ClN₄S: 379.0784 (MH+). Found 379.0772 (MH⁺).

### Example 14

### 4-[2-(4-trifluoromethoxyphenyl)pyridin-4-yl]-5-[6-methylpyridin-2-yl]-1,3-thiazol-2-amine

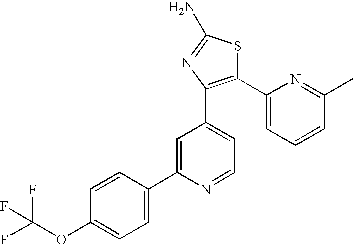

Intermediate 39 (0.372 g, 1 mmol) was reacted as described for example 13 to afford, after crystallisation from EtOH, the title compound as white crystals (0.17 g, 39.72%); m.p. 232° C.; TOF MS ES⁺ exact mass calculated for C₂₁H₁₅F₃N₄OS: 429.0997 (MH⁺). Found 429.0958 (MH+).

### Example 15

### 4-[2-(4(ethanesulfonyl)phenyl)pyridin-4-yl]-5-[6-methylpyridin-2-yl]-1,3-thiazol-2-amine

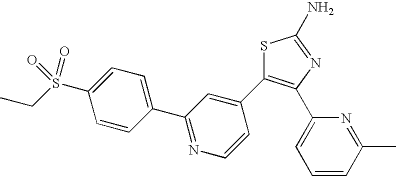

Intermediate 35 (0.321 g, 0.84 mmol) was reacted as described for example 13 to afford, after trituration with CH₂Cl₂, the title compound as a white solid (0.015 g, 4.1%); m.p. 219° C.; TOF MS ES⁺ exact mass calculated for C₂₂H₂₀N₄O₂S₂: 437.1106 (MH+). Found 437.1096 (MH+).

### Example 16

### 4-[2-(4-((tetrahydropyran-4-yl)-aminocarbonyl)phenyl)pyridin-4-yl]-5-[6-methylpyridin-2-yl]-1,3-thiazol-2-amine

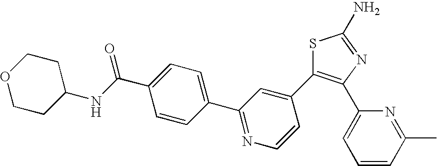

Intermediate 42 (0.8 g, 2 mmol) was reacted as described for example 13 to afford, after crystallisation from EtOH, the title compound as crystals.(0.202 g, 22.25%); m.p. 283° C.; TOF MS ES⁺ mass calculated for C₂₆H₂₅N₅O₂S: 472.1807 (MH+). Found 472.1815 (MH+).

### Example 17

### 4-[2-(4-((morpholin-4-yl)carbonyl)phenyl)pyridin-4-yl[-5-[6-methylpyridin-2-yl]-1,3-thiazol-2-amine

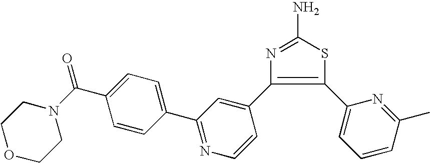

Intermediate 40 (0.6 g, 1.5 mmol) was reacted as described for example 13 to afford, after crystallisation EtOH, the title compound as yellow crystals (0.265 g, 38.66%); m.p. 246° C.; TOF MS ES⁺ exact mass calculated for C₂₅H₂₃N₅O₂S: 458.1651 (MH+). Found 458.1610 (MH+).

### Example 18

### 4-[2-(4-(1-ethyl-piperazin-4-yl)carbonyl)phenyl)pyridin-4yl]-5-[6-methylpyridin-2-yl]-1,3-thiazol-2-amine

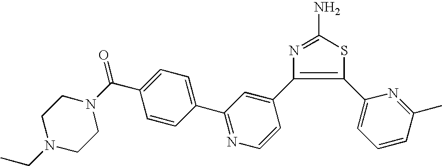

Intermediate 41 (0.428 g, 1 mmol) was reacted as described for example 13 to afford, after crystallisation from EtOH, the title compound as yellow crystals (0.2 g, 41.32%); m.p. 224° C.; TOF MS ES⁺ exact mass calculated for C₂₇H₂₈N₆OS: 485.2123 (MH+). Found 485.2128 (MH+).

### Example 19

### 4-[2-(4-((morpholin-4-yl)methyl)phenyl)pyridin-4-yl]-5-[6-methylpyridin-2-yl]-1,3-thiazol-2-amine

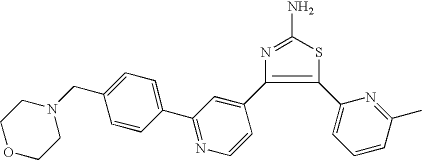

Intermediate 48 (0.58 g, 1.5 mmol) was reacted as described for example 13 to afford, after crystallisation from EtOH, the title compound as yellow crystals (0.33 g, 50%); m.p. 236° C.; TOF MS ES⁺ exact mass calculated for C₂₅H₂6N₅OS: :444.1858 (MH+). Found 444.1862 (MH+).

### Example 20

### 4-[2-(4-(morpholin-4-yl)phenyl)pyridin-4-yl]-5-[6-methylpyridin-2-yl]-1,3-thiazol-2-amine

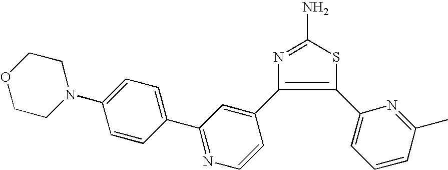

Intermediate 43 (0.379 g, 1 mmol) was reacted as described for example 13 to afford, after crystallisation from acetonitrile, the title compound as yellow crystals (0.148 g, 34%); m.p. 246° C.; TOF MS ES⁺ exact mass calculated for C₂₄H₂₃N₅OS: 430.1701 (MH+). Found 430.1648 (MH+).

### Example 21

### 4-[2-(4-(2-(pyrolidin-1-yl)-ethoxy)phenyl)pyridin-4-yl]-5-[6-methylpyridin-2-yl]-1,3-thiazol-2-amine

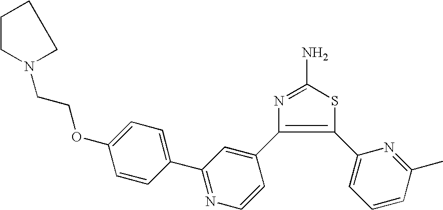

Intermediate 44 (0.4 g, 1 mmol) was reacted as described for example 13 to afford, after crystallisation from ethyl acetate, the title compound as white crystals (0.225 g, 49.36%); m.p. 150° C.; TOF MS ES⁺ exact mass calculated for C₂₆H₂₇N₅OS: 458.2014 (MH+). Found 458.1963 (MH+).

### Example 22

### 4-[2-(4-(aminocarbonylmethyloxy)phenyl)pyridin-4-yl]-5-[6-methylpyridin-2-yl]-1,3-thiazol-2-amine

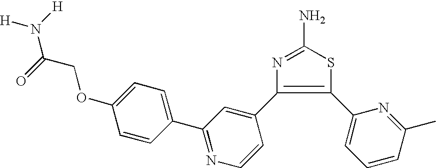

Intermediate 45 (0.542 g, 1.5 mmol) was reacted as described for example 13 to afford, after crystallisation from EtOH, the title compound as yellow crystals (0.14 g, 22.36%); m.p. 191° C.; TOF MS ES⁺ exact mass calculated for C₂₂H₁₉N₅O₂S: 418.1338 (MH+). Found 418.1289 (MH+).

### Example 23

### 4-[2-(4-((morpholin-4-yl)carbonylmethyloxy)phenyl)pyridin-4-yl]-5-[6-methylpyridin-2-yl]-1,3-thiazol-2-amine

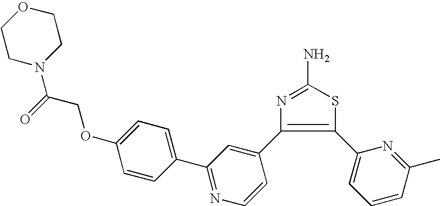

Intermediate 46 (0.431 g,1 mmol) was reacted as described for example 13 to afford, after crystallisation from toluene, the title compound as white crystals (0.26 g, 53.39%); m.p. 172° C.; TOF MS ES⁺ exact mass calculated for C₂₆H₂₅N₅O₃S (MH+) calculated 488.1756 (MH+). Found 488.1700 (MH+).

### Example 24

### 4-[2-(4-((pyrrolidin-1-yl)methyl)phenyl)pyridin-4-yl]-5-[6-methylpyridin-2-yl]-1,3-thiazol-2-amine

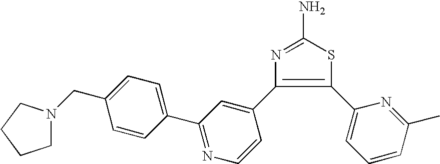

Intermediate 49 (0.5 g, 1.35 mmol) was reacted as described for example 13 to afford, after crystallisation from EtOH, the title compound as crystals (0.194 g, 33.71%); m.p. 200° C.; TOF MS ES⁺ exact mass calculated for C₂₅H₂₅N₅S (MH+) calculated 428.1909 (MH+). Found 428.1861 (MH+).

### Example 25

### 4-[2-(4-((dimethylamino)methyl)phenyl)pyridin-4-yl]-5-[6-methylpyridin-2-yl]-1,3-thiazol-2-amine

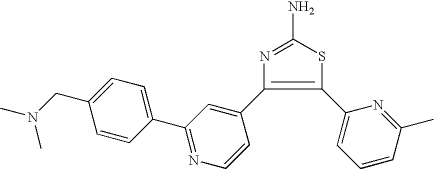

Intermediate 50 (0.4 g, 1.16 mmol) was reacted as described for example 13 to afford, after crystallisation from EtOH, the title compound as crystals (0.176 g, 37.86%); m.p. 210° C.; TOF MS ES⁺ exact mass calculated for C₂₃H₂₃N₅S (MH+) calculated 402.1752 (MH+). Found 402.1707 (MH+).

### Example 26

### 4-[2-(4-((tetrahydropyran-4-yl)aminocarbonyl)phenyl)pyridin-4-yl]-5-[pyridin-2-yl]-1,3-thiazol-2-amine

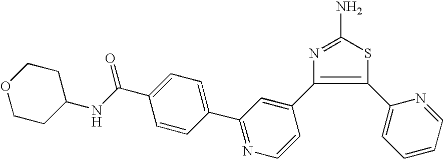

Intermediate 52 (0.4 g, 1.16 mmol) was reacted as described for example 13 to afford, after crystallisation from EtOH, the title compound as yellow crystals (0.395 g, 43.32%); m.p. 268° C.; TOF MS ES⁺ exact mass calculated for C₂₅H₂₃N₅O₂S (MH+) calculated 458.1651 (MH+). Found 458.1637 (MH+).

### Example 27

### 4-[2-(4-(morpholin-4-yl)phenyl)pyridin-4-yl]-5-[pyridin-2-yl]-1,3-thiazol-2-amine

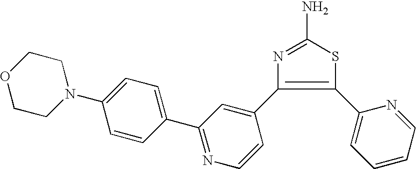

Intermediate 53 (0.6 g, 1.67 mmol) was reacted as described for example 13 to afford, after crystallisation from EtOH, the title compound as yellow crystals (0.179 g, 25.81%); m.p. 276° C.; TOF MS ES⁺ exact mass calculated for C₂₃H₂₁N₅OS: calculated 416.1545 (MH+). Found 416.1504 (MH+).

### Examples 28 to 30

Step 1: Intermediate 54 supported on resin (1 g) was weighed out into a peptide vessel. Then 4-formylphenylboronic acid (870 mg, 5.8 mmol, 10 eq), Pd(PPh₃)₄ (134 mg, 0.16 mmol, 0.2 eq), and sodium carbonate (615 mg, 5.8 mmol, 2M) were added and suspended in toluene/EtOH (8:2, 20 mL). The reaction vessel was purged with argon for 5 min, and the mixture was stirred at 90° C. for 16 h. The resin was washed with DMF (3×10 mL), water (3×10 mL), EtOH (3×10 mL) and CH₂Cl₂ (3×10 mL).

Step 2: The product from step 1 was placed into a peptide vessel with a solution of NHR⁵R⁶ (5.8 mmol, 10 eq) in tnmethylorthoformate (5.4 mL). Then a solution of sodium cyanoborohydride (0.2M) in THF (5.4 mL) with acetic acid (110 μL) was added. The reaction vessel was purged with argon for 5 min and the mixture was stirred at 60° C. for 16 h. The resin was washed with DMF (3×10 mL), EtOH (3×10 mL) and CH₂Cl₂ (3×10 mL). The resin was treated with a solution of 20% TFA in CH₂Cl₂ and the solvent was removed under reduced pressure. Purification of the residue by HPLC chromatography (water/acetonitrile gradient) gave the products of formula (Id′) shown in Table 1, i.e. compounds of general formula (I) where A is S, B is N, R² and R³ are hydrogen and R¹ is —CH₂NR⁵R⁶.

TABLE 1

| | |
|---|---|
| | (Id′) |
| 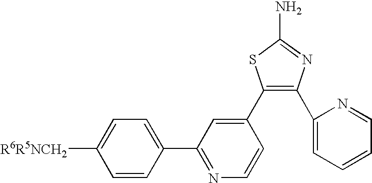 | |

| Ex | R⁵ | R⁶ | Physical data |
|---|---|---|---|
| 28 | H | isopropyl | [APCI MS] m/z 402 MH+ |
| 29 | —(CH₂)₄— | | TOF MS ES⁺ exact mass calculated |
| | | | for C₂₄H₂₃N₅S |
| | | | (MH+): 414.1752. Found: 414.1766. |
| 30 | H | cyclobutyl | TOF MS ES⁺ exact mass calculated |
| | | | for C₂₄H₂₃N₅S |
| | | | (MH+): 414.1752. Found: 414.1749. |

### Examples 31 to 34

Step 1: Intermediate 54 or intermediate 55 supported on resin (1 g) were weighed out into a peptide vessel. Then 4-hydroxyphenylboronic acid (800 mg, 5.8 mmol, 10 eq), Pd(PPh₃)₄ (134 mg, 0.16 mmol, 0.2 eq), and sodium carbonate (615 mg, 5.8 mmol, 2M) were added and suspended in toluene/EtOH (8:2, 20 mL).The reaction vessel was purged with argon for 5 min, and the mixture was stirred at 90° C. for 16 h. The resin was washed with DMF (3×10 mL), water (3×10 mL), EtOH (3×10 mL) and CH₂Cl₂ (3×10 mL).

Step 2: The product from step 1 was placed into a peptide vessel with a solution of R—Cl (5.8 mmol, 10 eq) in DMSO (10 mL). Then a solution of potassium carbonate (802 mg, 5.8 mmol, 10 eq) in DMSO (5 mL) was added. The reaction vessel was purged with argon for 5 min and the mixture was stirred at 90° C. for 16 h. The resin was washed with DMF (3×10 mL), EtOH (3×10 mL) and CH₂Cl₂ (3×10 mL). The resin was treated with a solution of 20% TFA in CH₂Cl₂ and the solyent was remoyed under reduced pressure. Purification of the residue by HPLC chromatography (water/acetonitrile gradient) gave the products of formula (Ic′) shown in Table 2, i.e. compounds of general formula (I) where A is S, B is N, R³ is hydrogen, R⁴ is NH₂ and R¹ is OR.

TABLE 2

| | |
|---|---|
| | (Ic′) |
| 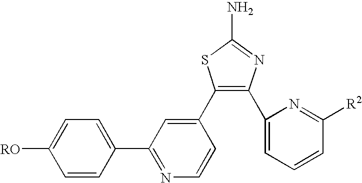 | |

| | | | From intermediate | |
|---|---|---|---|---|
| Ex | R | R² | supported on resin | Physical data |
| 31 | 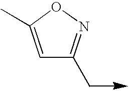 | methyl | 55 | TOF MS ES⁺ exact mass calculated for C₂₅H₂₁N₅O₂S (MH+): 456.1494. Found: 456.1457. |
| 32 | 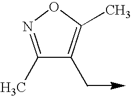 | H | 54 | TOF MS ES⁺ exact mass calculated for C₂₅H₂₁N₅O₂S (MH+): 456.1494. Found: 456.1545. |
| 33 | 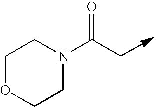 | methyl | 55 | TOF MS ES⁺ exact mass calculated for C₂₆H₂₅N₅O₃S (MH+): 488.1756. Found: 488.1792. |
| 34 | 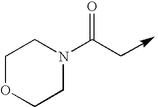 | H | 54 | TOF MS ES⁺ exact mass calculated for C₂₅H₂₃N₅O₃S (MH+): 474.1600. Found: 474.1552. |

### Examples 35 to 46

Step 1: Intermediate 55 supported on resin (1 g) was weighed out into a peptide vessel. Then 4-methoxycarbonylphenylboronic acid (1.05 g, 5.8 mmol, 10 eq), Pd(PPh₃)₄ (0.134 g, 0.16 mmol, 0.2 eq), and a aqueous solution of sodium carbonate (0.615 g, 5.8 mmol, 2M) were added and suspended in toluene/EtOH (8:2, 20 mL). The reaction vessel was purged with argon for 5 min, and the mixture was stirred at 90° C. for 16 h. The resin was washed with DMF (3×10 mL), water (3×10 mL), EtOH (3×10 mL) and CH₂Cl₂ (3×10 mL). Then resin was added to a sodium hydroxide solution (2M) in dioxane (10 mL). The reaction mixture was stirred at 50° C. for 16 h. The resin was washed with DMF (3×10 mL), EtOH (3×10 mL) and CH₂Cl₂ (3×10 mL).

Step 2: The product from step 1 was placed into a peptide vessel with a solution of NHR⁵R⁶ (5.8 mmol, 10 eq) in DMF (5 mL). Then a solution of HOBT (1.18 g, 8.7 mmol, 15 eq) and EDCl (1.36 mL, 8.7 mmol, 15 eq) in DMF (5 mL) was added. The reaction vessel was purged with argon for 5 min and the mixture was stirred at 70° C. for 16 h. The resin was washed with DMF (3×10 mL), EtOH (3×10 mL), CH₂Cl₂ (3×10 mL). The resin was treated with a solution of 20% TFA in CH₂Cl₂ and the solyent was removed under reduced pressure. Purification of the residue by HPLC chromatography (water/acetonitrile gradient) gave the products of formula (Ie′) shown in Table 3, i.e. compounds of general formula (I) where A is S, B is N, R² is methyl, R³ is hydrogen, and R¹ is —CONR⁵R⁶.

TABLE 3

| | |
|---|---|
| | (Ie′) |
| 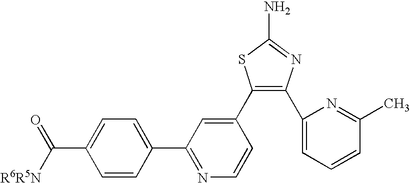 | |

| Ex | R⁶R⁵NCO— | Physical data |
|---|---|---|
| 35 | 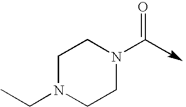 | TOF MS ES⁺ exact mass calculated for C₂₇H₂₈N₆OS (MH+): 485.2123. Found: 485.2123. |
| 36 | 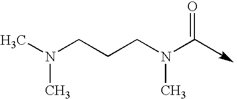 | TOF MS ES⁺ exact mass calculated for C₂₇H₃₀N₆OS (MH+): 487.2280. Found: 487.2267. |
| 37 | 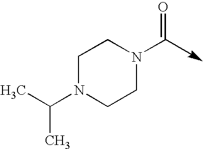 | TOF MS ES⁺ exact mass calculated for C₂₈H₃₀N₆OS (MH+): 499.2280. Found: 499.2277. |
| 38 | 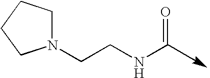 | TOF MS ES⁺ exact mass calculated for C₂₇H₂₈N₆OS (MH+): 485.2123. Found: 485.2094. |
| 39 | 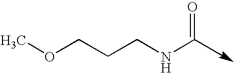 | TOF MS ES⁺ exact mass calculated for C₂₅H₂₅N₅O₂S (MH+): 460.1807. Found: 460.1810. |
| 40 | 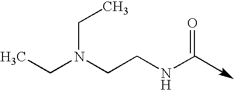 | TOF MS ES⁺ exact mass calculated for C₂₇H₃₀N₆OS (MH+): 487.2280. Found: 487.2260. |
| 41 | 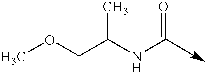 | TOF MS ES⁺ exact mass calculated for C₂₅H₂₅N₅O₂S (MH+): 460.1807. Found: 460.1799. |
| 42 | 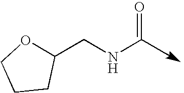 | TOF MS ES⁺ exact mass calculated for C₂₆H₂₅N₅O₂S (MH+): 472.1807. Found: 472.1798. |
| 43 | 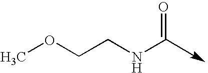 | TOF MS ES⁺ exact mass calculated for C₂₄H₂₃N₅O₂S (MH+): 466.1651. Found: 466.1633. |
| 44 | 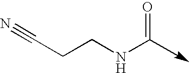 | TOF MS ES⁺ exact mass calculated for C₂₅H₂₂N₆OS (MH+): 455.1654. Found: 455.1626. |
| 45 | 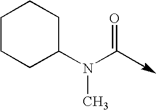 | TOF MS ES⁺ exact mass calculated for C₂₈H₂₉N₅OS (MH+): 484.2171. Found: 484.2133. |
| 46 | 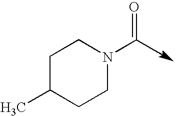 | TOF MS ES⁺ exact mass calculated for C₂₇H₂₇N₅OS (MH+): 470.2014. Found: 470.1964. |

Biology

The biological activity of the compounds of the invention may be assessed using the following assays:

Assay 1 (Cellular Transcriptional Assay)

The potential for compounds of the invention to inhibit TGF-β signalling may be demonstrated, for example, using the following in vitro assay.

The assay was performed in HepG2 cells stably transfected with the PAI-1 promoter (known to be a strong TGF-β responsive promoter) linked to a luciferase (firefly) reporter gene. The compounds were selected on their ability to inhibit luciferase activity in cells exposed to TGF-β. In addition, cells were transfected with a second luciferase (Renilla) gene which was not driven by a TGF-β responsive promoter and was used as a toxicity control.

96 well microplates were seeded, using a multidrop apparatus, with the stably transfected cell line at a concentration of 35000 cells per well in 200 μl of serum-containing medium. These plates were placed in a cell incubator.

18 to 24 hours later (Day 2), cell-incubation procedure was launched. Cells were incubated with TGF-β and a candidate compound at concentrations in the range 50 nM to 10 μM (final concentration of DMSO 1%). The final concentration of TGF-β (rhTGFβ-1) used in the test was 1 ng/mL. Cells were incubated with a candidate compound 15-30 mins prior to the addition of TGF-β. The final volume of the test reaction was 150 μl. Each well contained only one candidate compound and its effect on the PAI-1 promoter was monitored.

Columns 11 and 12 were employed as controls. Column 11 contained 8 wells in which the cells were incubated in the presence of TGF-β, without a candidate compound. Column 11 was used to determine the ‘reference TGF-β induced firefly luciferase value’ against which values measured in the test wells (to quantify inhibitory activity) were compared. In wells A12 to D12, cells were grown in medium without TGF-β. The firefly luciferase values obtained from these positions are representative of the ‘basal firefly luciferase activity’. In wells E12 to H12, cells were incubated in the presence of TGF-β and 500 μM CPO (Cyclopentenone, Sigma), a cell toxic compound. The toxicity was revealed by decreased firefly and renilla luciferase activities (around 50% of those obtained in column 11).

12 to 18 hours later (day 3), the luciferase quantification procedure was launched. The following reactions were performed using reagents obtained from a Dual Luciferase Assay Kit (Promega). Cells were washed and lysed with the addition of 10 μl of passive lysis buffer (Promega). Following agitation (15 to 30 mins), luciferase activities of the plates were read in a dual-injector luminometer (BMG lumistar). For this purpose, 50 μl of luciferase assay reagent and 50 μl of ‘Stop & Glo’ buffer were injected sequentially to quantify the activities of both luciferases. Data obtained from the measurements were processed and analysed using suitable software. The mean Luciferase activity value obtained in wells A11 to H11 (Column 11, TGF-βonly) was considered to represent 100% and yalues obtained in wells A12 to D12 (cells in medium alone) gave a basal level (0%). For each of the compounds tested, a concentration response curve was constructed from which an IC₅₀ value was determined graphically.

Assay 2 (Alk5 Fluorescence Polarization Assay)

Kinase inhibitor compounds conjugated to fluorophores, can be used as fluorescent ligands to monitor ATP competitive binding of other compounds to a given kinase. The increase in depolarization of plane polarized light, caused by release of the bound ligand into solution, is measured as a polarization/anisotropy value. This protocol details the use of a rhodamine green-labelled ligand for assays using recombinant GST-ALK5 (residues 198-503).

Assay buffer components: 62.5 mM Hepes pH 7.5 (Sigma H-4034), 1 mM DTT (Sigma D-0632), 12.5 mM MgCl₂ (Sigma M-9272), 1.25 mM CHAPS (Sigma C-3023).

Protocol: Solid compound stocks were dissolved in 100% DMSO to a concentration of 1 mM and transferred into column 1, rows A-H of a 96-well, U bottom, polypropylene plate (Costar #3365) to make a compound plate. The compounds were serially diluted (3-fold in 100% DMSO) across the plate to column 11 to yield 11 concentrations for each test compound. Column 12 contained only DMSO. A Rapidplate™-96 was used to transfer 1 μl of sample from each well into a 96-well, black, U-bottom, non-treated plate (Costar #3792) to create an assay plate.

ALK5 was added to assay buffer containing the above components and 1 nM of the rhodamine green-labelled ligand so that the final ALK5 concentration was 10 nM based on active site titration of the enzyme. The enzyme/ligand reagent (39 μl) was added to each well of the previously prepared assay plates. A control compound (1 μl) was added to column 12, rows E-H for the low control values. The plates were read immediately on a LJL Acquest fluorescence reader (Molecular Devices, serial number AQ1048) with excitation, emission, and dichroic filters of 485 nm, 530 nm, and 505 nm, respectively. The fluorescence polarization for each well was calculated by the Acquest reader and then imported into curve fitting software for construction of concentration response curves. The normalized response was determined relative to the high controls (1 μl DMSO in column 12, rows A-D) and the low controls (1 μl of control compound in column 12, rows E-H). An IC₅₀ value was then calculated for each compound

Using the above assays all Examples of the invention show ALK5 receptor modulator activity (having IC₅₀ values in the range of 1 to 300 nM) and TGF-β cellular activity (having IC₅₀ values in the range of 0.001 to 10 μM).

5-{2-[4-(4-Ethylpiperazin-1-yl)phenyl]pyridin-4-yl}-4-(6-methylpyridin-2-yl)-1,3-thiazol-2-amine (Example 3) showed an ALK5 receptor modulator activity of 14 nM and TGF-β cellular activity of 29 nM.

4-[2-(4-((Dimethylamino)methyl)phenyl)pyridin-4-yl]-5-[6-methylpyridin-2-yl]-1,3-thiazol-2-amine (Example 25) showed an ALK5 receptor modulator activity of 29 nM and TGF-β cellular activity of 31 nM.

1. A compound of formula (I), a pharmaceutically acceptable salt, solvate or derivative thereof: 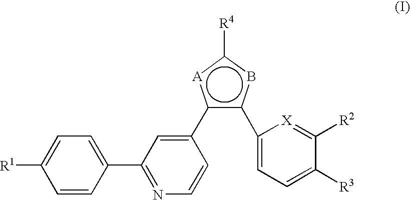
wherein
either A is S and B is N, or A is N and B is S;
X is N or CH;
R¹ is selected from hydrogen, C₁₋₆alkyl, C₁₋₆alkenyl, C₁₋₆alkoxy, halo, cyano, perfluoro C₁₋₆alkyl, perfluoroC₁₋₆alkoxy, —NR⁵R⁶, —(CH₂)ₙNR⁵R⁶, —O(CH₂)ₙOR⁷, —O(CH₂)ₙ-Het, —O(CH₂)ₙNR⁵R⁶, —CONR⁵R⁶, —CO(CH₂)ₙNR⁵R⁶, —SO₂R⁷, —SO₂NR⁵R⁶, —NR⁵SO₂R⁷, —NR⁵COR⁷ and —O(CH₂)ₙCONR⁵R⁶;
R² is hydrogen, C₁₋₆alkyl, halo, cyano or perfluoroC₁₋₆alkyl;
R³ is hydrogen or halo;
R⁴is —NH₂;
where
R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵ and R⁶ together with the nitrogen atom to which they are attached form a 3, 4, 5, 6 or 7-membered saturated or unsaturated ring which may contain one or more heteroatoms selected from N, S or O, and wherein the ring may be further substituted by one or more substituents selected from halo (such as fluoro, chloro, bromo), cyano, —CF₃, hydroxy, —OCF₃, C₁₋₆alkyl and C₁₋₆alkoxy;
R⁷ is hydrogen or C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4;
with the proviso that the compound of formula (I) is not:
5-[2-(4-chlorophenyl)pyridin-4-yl]-4-pyridin-2-yl-1,3-thiazol-2-amine;
5-[2-(4-methoxyphenyl)pyridin-4-yl]-4-pyridin-2-yl-1,3-thiazol-2-amine;
5-[2-(4-fluorophenyl)pyridin-4-yl]-4-pyridin-2-yl-1,3-thiazol-2-amine;
5-[2-(4-ethylphenyl)pyridin-4-yl]-4-pyridin-2-yl-1,3-thiazol-2-amine; or
5-[2-(4-ethoxyphenyl)pyridin-4-yl]-4-pyridin-2-yl-1,3-thiazol-2-amine.

2. A compound according to claim 1 with the proviso that when A is S; B is N; X is N; R¹ is hydrogen, C₁₋₆alkyl, C₁₋₆alkoxy, halo, cyano, perfluoroC₁₋₆alkyl or perfluoroC₁₋₆alkoxy; R² is hydrogen, C₁₋₆alkyl, halo, cyano or perfluoroC₁₋₆alkyl; and R³ is hydrogen or halo; then R⁴ is not NH₂.

3. A compound according to claim 1 wherein X is N.

4. A compound according to claim 1 wherein R¹ is —NR⁵R⁶, —(CH₂)ₙNR⁵R⁶, —O(CH₂)ₙ-Het, —O(CH₂)ₙNR⁵R⁶, —CONR⁵R⁶, —SO₂R⁷ or —O(CH₂)ₙCONR⁵R⁶.

5. A compound according to claim 1 wherein R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy.

6. A compound according to claim 1 wherein R¹ is morpholin-4-yl, methanesulfonyl, 4-ethylpiperazin-1-yl, (morpholin-4-yl)carbonyl, (tetrahydropyran-4-yl)-aminocarbonyl, (morpholin-4-yl)methyl, aminocarbonylmethyloxy, 2-(pyrrolidin-1-yl)-ethoxy, (1-methyl-imidazol-4-yl)methyloxy, ethanesulfonyl, 4-(1-ethyl-piperazin-4-yl)carbonyl, (morpholin-4-yl)carbonylmethyloxy, (pyrolidin-1-yl)methyl, (dimethylamino)methyl, isopropylaminomethyl, cyclobutylaminomethyl, (5-methyl-isoxazol-3-yl)methoxy, (3,5-dimethylisoxazol-4-yl)methoxy, N-methyl-N-(3-dimethylaminopropyl)aminocarbonyl, 4-(1-isopropyl-piperazin-4-yl)carbonyl, 2-(pyrrolidin-1-yl)ethylaminocarbonyl, 3-methoxypropylaminocarbonyl, 2-(diethylamino)ethylaminocarbonyl, (2-methoxy-1-methyl)ethylaminocarbonyl, (tetrahydrofuran-2-yl)methylaminocarbonyl, 2-methoxyethylaminocarbonyl, 2-cyanoethylaminocarbonyl, (N-methyl-N-cyclohexyl)aminocarbonyl or 4-methyl-piperidin-1-ylcarbonyl.

7. A compound according to claim 6 wherein R¹ is (tetrahydropyran-4-yl)-aminocarbonyl, (pyrolidin-1-yl)methyl, (dimethylamino)methyl, (morpholin-4-yl)methyl, morpholin-4-yl, 4-ethylpiperazin-1-yl or aminocarbonylmethyloxy.

8. A compound according to claim 1 wherein R² is hydrogen, C₁₋₆alkyl, chloro or fluoro.

9. A compound according to claim 1 wherein R³ is hydrogen or fluoro.

10. A compound according to claim 1 wherein when X is N, R² is methyl.

11. A compound according to claim 1 wherein when X is N and R² is methyl, R³ is hydrogen.

12. A compound according to claim 1 wherein
either A is S and B is N, or A is N and B is S;
X is N;
R¹ is —NR⁵R⁶, —(CH₂)ₙNR⁵R⁶, —O(CH₂)ₙ-Het, —O(CH₂)ₙNR⁵R⁶, —CONR⁵R⁶, —SO₂R⁷ or —O(CH₂)ₙCONR⁵R⁶;
R²is hydrogen, methyl, chloro or fluoro;
R³ is hydrogen or halo;
R⁴is —NH₂;
where
R⁵ and R⁶ are independently selected from hydrogen; Het; C₃₋₆cycloalkyl optionally substituted by C₁₋₆alkyl; or by C₁₋₆alkyl optionally substituted by Het, alkoxy, cyano or —NR^{a}R^{b} (where R^{a} and R^{b} which may the same or different are hydrogen or C₁₋₆alkyl, or R^{a} and R^{b} together with the nitrogen atom to which they are attached may form a 4, 5 or 6-membered saturated ring); or R⁵ and R⁶ together with the atom to which they are attached form a morpholine, piperidine, pyrrolidine or piperazine ring, each of which may be substituted by halo (such as fluoro, chloro, bromo), cyano, —CF₃, hydroxy, —OCF₃, C₁₋₄alkyl or C₁₋₄alkoxy;
R⁷ is hydrogen or C₁₋₆alkyl;
Het is a 5 or 6-membered C-linked heterocyclyl group which may be saturated, unsaturated or aromatic, which may contain one or more heteroatoms selected from N, S or O and which may be substituted by C₁₋₆alkyl; and
n is 1-4.

13. A compound according to claim 1 selected from the list:
5-{2-[4-(4-ethylpiperazin-1-yl)phenyl]pyridin-4-yl}-4-(6-methylpyridin-2-yl)-1,3-thiazol-2-amine (Example 3);
5-{2-[4-(morpholin-4-yl)phenyl]pyridin-4-yl}-4-(6-methylpyridin-2-yl)-1,3-thiazol-2-amine (Example 4);
5-{2-[4-(aminocarbonylmethyloxy)-phenyl]pyridin-4-yl}-4-(6-methylpyridin-2-yl)-1,3-thiazol-2-amine (Example 10);
5-{2-[4-(2-(pyrrolidin-1-yl)-ethoxy)-phenyl]pyridin-4-yl}-4-(6-methylpyridin-2-yl)-1,3-thiazol-2-amine (Example 11);
4-[2-(4-((tetrahydropyran-4-yl)-aminocarbonyl)phenyl)pyridin-4-yl]-5-[6-methylpyridin-2-yl]-1,3-thiazol-2-amine (Example 16);
4-[2-(4-(morpholin-4-yl)phenyl)pyridin-4-yl]-5-[6-methylpyridin-2-yl]-1,3-thiazol-2-amine (Example 20);
4-[2-(4-(aminocarbonylmethyloxy)phenyl)pyridin-4-yl]-5-[6-methylpyridin-2-yl]-1,3-thiazol-2-amine (Example 22);
4-[2-(4-((pyrrolidin-1-yl)methyl)phenyl)pyridin-4-yl]-5-[6-methylpyridin-2-yl]-1,3-thiazol-2-amine (Example 24);
4-[2-(4-((dimethylamino)methyl)phenyl)pyridin-4-yl]-5-[6-methylpyridin-2-yl]-1,3-thiazol-2-amine (Example 25); and
4-[2-(4-((tetrahydropyran-4-yl)aminocarbonyl)phenyl)pyridin-4-yl]-5-[pyridin-2-yl]-1,3-thiazol-2-amine (Example 26);
and pharmaceutically acceptable salts, solvates and derivatives thereof.

14. A pharmaceutical composition comprising a compound defined in claim 1 and a pharmaceutically acceptable carrier or diluent.

15-18. (canceled)

19. A method for the treatment or prophylaxis of a disorder mediated by the ALK5 receptor in mammals, wherein the disorder is selected from chronic renal disease, acute renal disease, wound healing, arthritis, osteoporosis, kidney disease, congestive heart failure, ulcers, ocular disorders, corneal wounds, diabetic nephropathy, impaired neurological function, Alzheimer's disease, atherosclerosis, peritoneal and sub-dermal adhesion, any disease wherein fibrosis is a major component, including, but not limited to lung fibrosis, kidney fibrosis, liver fibrosis [for example, hepatitis B virus (HBV), hepatitis C virus (HCV)], alcohol induced hepatitis, retroperitoneal fibrosis, mesenteric fibrosis, haemochromatosis and primary biliary cirrhosis, endometriosis, keloids and restenosis, which method comprises administering to a mammal in need of such treatment a compound of formula I.
